(19) United States
(12) Patent Application Publication
(10) Pub. No.: US 2002/0102563 A1
(43) Pub. Date: Aug. 1, 2002

(54) Apparatus and method for separating and purifying polynucleotides

(76) Inventors: Douglas T. Gjerde, Saratoga, CA (US); Christopher P. Hanna, Greenfield, MA (US); David Hornby, Cheshire (GB); Mark Dickman, Broomhill (GB); Benjamin L. Legendre JR., Bellevue, NE (US); Paul D. Taylor, Gilroy, CA (US); Robert M. Haefele, Campbell, CA (US); Arezou Azarani, San Jose, CA (US)

Correspondence Address: WILLIAM B. WALKER, TRANSGENOMIC, INC., 2032 CONCOURSE DRIVE, SAN JOSE, CA, 95131, US

(21) Appl. No.: 09/809,867

(22) Filed: Mar. 15, 2001

Related U.S. Application Data
(63) Continuation-in-part of application No. 09/318,407, filed on May 25, 1999.
(63) Continuation-in-part of application No. 09/164,041, filed on Sep. 30, 1998.
Application No. 09/164,041 is a continuation-in-part of application No. 09/391,963, filed on Sep. 8, 1999.
Application No. 09/391,963 is a continuation of application No. 09/065,913, filed on Apr. 24, 1998.
(63) Continuation-in-part of application No. 09/557,424, filed on Apr. 21, 2000.
(60) Provisional application No. 60/129,838, filed on Apr. 16, 1999.
(60) Provisional application No. 60/103,743, filed on Oct. 9, 1998.
(60) Provisional application No. 60/187,979, filed on Mar. 9, 2000.
(60) Provisional application No. 60/187,974, filed on Mar. 9, 2000.

(51) Int. Cl. C12Q 1/68; C07H 21/04

(52) U.S. Cl. 435/6; 536/25.4; 210/656

(57) ABSTRACT

The instant invention provides a non-HPLC chromatographic method for purifying a target polynucleotide comprising the steps of: applying the target polynucleotide to a separation medium having a non-polar separation surface in the presence of a counterion agent, whereby the polynucleotide is bound to the separation medium; eluting the target polynucleotide from the separation medium by passing through the separation medium an elution solution containing a concentration of organic solvent sufficient to elute the target polynucleotide from the separation medium; and collecting the eluted target polynucleotide. The separation medium can be supported in any of a variety of containers, non-limiting preferred examples of which include spin columns and vacuum trays. The invention is particularly useful for the separation of RNA and single and double stranded DNA. In preferred embodiments of the invention the purification is accomplished under conditions that are substantially free of multivalent cations capable of interfering with polynucleotide separations.

### RELATIONSHIP TO COPENDING APPLICATIONS

This application is a continuation-in-part of the following U.S. patent applications:

U.S. patent application No. 09/318,407, filed May 25, 1999, which claims priority to U.S. Provisional Patent Application No. 60/129,838, filed Apr. 16, 1999 and U.S. Provisional Patent Application No. 60/103,743, filed Oct. 9,1998;

U.S. patent application No. 09/164,041, filed Jan. 16, 2001, which is a continuation-in-part of U.S. patent application No. 09/391,963, filed Sep. 8, 1999, which is a continuation of U.S. patent application No. 09/065,913, filed Apr. 24,1998; and

U.S. patent application No. 09/557,424, filed Apr. 21, 2000, which claims priority to U.S. Provisional Patent Application Nos. 60/187,979 and 60/187,974, both filed Mar. 9, 2000.

The entire contents of the above-listed pending patent applications are hereby incorporated by reference into the present application.

### FIELD OF THE INVENTION

This invention relates to an apparatus and method that can be used for separating, isolating, and purifying polynucleotides, including single-stranded and double-stranded DNA and RNA. In some embodiments, this invention relates to methods and devices for separating target polynucleotides having a predetermined size or range of sizes.

### BACKGROUND OF THE INVENTION

The separation and purification of polynucleotides such as DNA (both double and single stranded) and RNA is of critical importance in molecular biology, and improved methods are a focus of current interest. A variety of methodologies have been developed for achieving these separations. Traditionally such separation techniques have often relied on gel electrophoresis. For example, a polynucleotide of interest can be purified from a sample by gel electrophoresis of the sample followed by physical excision of the band corresponding to the polynucleotide (e.g., cutting out the band and recovery from low melting temperature agarose, electroelution, electrophoresis onto NA-45 paper (Schleicher and Schuell)). Disadvantages of gel based techniques include the time and effort required for sample preparation, gel preparation, electrophoresis, band detection, band excision/recovery, and post-excision clean-up. These disadvantages can be particularly burdensome where the high-throughput processing of multiple samples is desired. Furthermore, polynucleotides can become covalently modified by the chemicals used during the fractionation process (e.g., formaldehyde or acrylamide), and these techniques often involve the used of hazardous chemicals (e.g., acrylamide, ethidium bromide, methylmercuric hydroxide). A number of gel electrophoresis separation and purification techniques are well known in the art and are described, for example, in Molecular Cloning: a Laboratory Manual: 2nd edition, 3 Volumes, Sambrook et al, 1989, Cold Spring Harbor Laboratory Press (or later editions of the same work) or Current Protocols in Molecular Biology, Second Edition, Ausubel et al. eds., John Wiley & Sons, 1992.

In addition, a number of chromatographic techniques have been developed for the separation and purification of polynucleotides. One example is size-exclusion chromatography (E. Heftmann, in J. Chromatog. Lib., Vol. 51A, p. A299 (1992)). However, disadvantages of this method include low resolution and low capacity. Another separation method, anion exchange chromatography of DNA with a mobile phases containing tetramethylammonium chloride is described in European patent application 0 507 591 A2 to Bloch. However, this method of separation is not strictly size-based, and the resolution is not always adequate. A further disadvantage of methods that rely on binding of anionic DNA is the required use of high concentrations of nonvolatile salts in the mobile phase; this interferes with subsequent isolation and measurement (e.g. mass spectrometry analysis) on the separated fragments.

In the preparation of mRNA from total RNA, spin columns containing beads coated with poly-T oligomers are often used (e.g., Poly(A)Pure™ mRNA Purification Kit, Ambion, Inc., Austin, Tex.; Oligotex™ mRNA Purification System, Qiagen, Inc., Valencia, Calif.). The disadvantages of this technique include a requirement for high amounts of total RNA sample due to low recovery of mRNA, contamination of the product (e.g. by rRNA), and degradation of the mRNA product.

There exists a need for methods and reagents capable of separating and purifying polynucleotides in a manner that avoids disadvantages and limitations inherent in the presently available systems. The instant invention addresses this need, as described below, and hence represents a substantial contribution to the field of molecular biology.

### SUMMARY OF THE INVENTION

The instant invention provides a non-HPLC chromatographic method for purifying a target polynucleotide or polynucleotides or separating a target polynucleotide or plurality of polynucleotides.

In one embodiment, the invention provides a non-HPLC chromatographic method of purifying a polynucleotide comprising the steps of applying the target polynucleotide to a separation medium having a non-polar separation surface in the presence of a counterion agent, whereby the polynucleotide is bound to the separation medium;eluting the target polynucleotide from the separation medium by passing through the separation medium an elution solution containing a concentration of organic solvent sufficient to elute the target polynucleotide from the separation medium; and collecting the eluted target polynucleotide.

In one embodiment of the invention, the target polynucleotide is applied to the separation medium as a component of a loading solution containing a non-target molecule.

In a preferred embodiment the non-target molecule is not bound to the separation medium in the presence of the loading solution, and is thereby eluted from the separation medium and separated from the target polynucleotide by passing the loading solution through the separation medium.

In another preferred embodiment of the invention the non-target molecule is bound to the separation medium in the presence of the loading solution, and including an additional step between steps (a) and (b) of eluting the non-target molecule from the separation medium by passing through the separation medium a wash solution containing a counterion agent and a concentration of organic solvent sufficient to elute the non-target molecule, but insufficient to elute the target polynucleotide from the separation medium, whereby the non-target molecule is separated from the target polynucleotide.

In another preferred embodiment the non-target molecule remains bound to the separation medium in the presence of the elution solution, and is thereby separated from the target polynucleotide during the elution step.

In particularly preferred embodiments of the separation medium has a nonpolar separation surface that is substantially free of multivalent cations that are capable of interfering with polynucleotide separations, and/or the solutions used are substantially free of multivalent cations capable of interfering with polynucleotide separations.

In a preferred embodiment of the invention the non-target molecule is a polynucleotide. In particularly preferred embodiments of the invention the polynucleotide is double-stranded DNA, RNA, or single-stranded DNA. The DNA can be an oligonucleotide.

In another embodiment of the invention a mixture of polynucleotide fragments of varying nucleotide length is applied to the separation medium, and the elution solution contains a concentration of organic solvent that has been predetermined to elute polynucleotide fragments falling within a defined range of nucleotide lengths, whereby polynucleotide fragments falling within the defined range of nucleotide lengths are eluted from the separation medium and thereby separated from other polynucleotides of the mixture. In preferred embodiments of the invention the polynucleotide fragments are double-stranded DNA fragments, single-stranded DNA fragments, or RNA fragments.

In a preferred embodiment of the invention the separation medium that is supported in a spin column. In this embodiment the separation medium is preferably in communication with an upper solution input chamber and a lower eluant receiving chamber, wherein the loading solution containing the polynucleotide and a counterion agent is applied to the separation medium by introducing the solution into the upper solution input chamber and centrifuging the spin column under conditions where the polynucleotide substantially binds to the separation medium, wherein the elution solution is passed through the separation medium by centrifugation of the spin column, and wherein the eluted polynucleotide is collected in the lower eluant receiving chamber.

In another preferred embodiment of the invention, the polynucleotide is eluted from separation medium that is supported in a vacuum tray separation device.

In some preferred embodiments of the invention the separation medium comprises particles selected from the group consisting of silica, silica carbide, silica nitrite, titanium oxide, aluminum oxide, zirconium oxide, carbon, insoluble polysaccharide, and diatomaceous earth, the particles having separation surfaces which are coated with a hydrocarbon or non-polar hydrocarbon substituted polymer, or have substantially all polar groups reacted with a non-polar hydrocarbon or substituted hydrocarbon group, wherein the surfaces are non-polar.

In other preferred embodiments of the invention, the separation medium comprises polymer beads having an average diameter of 0.5 to 100 microns, the beads being unsubstituted polymer beads or polymer beads substituted with a moiety selected from the group consisting of hydrocarbon having from one to 1,000,000 carbons.

In another preferred embodiment of the invention, the separation medium comprises a monolith.

In yet another preferred embodiment of the invention, the separation medium comprises capillary channels.

In a particularly preferred embodiment of the invention the separation medium has been subjected to acid wash treatment to remove any residual surface metal contaminants and/or has been subjected to treatment with a multivalent cation binding agent.

In some preferred embodiments of the invention, the organic solvent employed is selected from the group consisting of alcohol, nitrile, dimethylformamide, tetrahydrofuran, ester, ether, and mixtures of one or more thereof. A particularly preferred organic solvent comprises acetonitrile.

In some preferred embodiments of the invention, the counterion agent is selected from the group consisting of lower alkyl primary amine, lower alkyl secondary amine, lower alkyl tertiary amine, lower trialkylammonium salt, quaternary ammonium salt, and mixtures of one or more thereof. Particularly preferred counterion agents include octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyldiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, tetrabutylammonium acetate, triethylammonium hexafluoroisopropyl alcohol, and mixtures of one or more thereof. The most preferred counterion agents for use in the some aspects of the invention are tetrabutylammonium acetate and triethylammonium acetate.

In some embodiments of the invention the target polynucleotide is applied to the separation medium under denaturing conditions.

In a particularly preferred embodiment of the invention the method is used to separate a sample containing RNA and genomic into a RNA-containing fraction and a genomic DNA-containing fraction.

Another aspect of the invention is a device for purifying a target polynucleotide comprising a tube having: an upper solution input chamber; a lower eluant receiving chamber; and a fixed unit of separation medium supported therebetween, wherein the separation medium has a nonpolar separation surface that is substantially free of multivalent cations that are capable of interfering with polynucleotide separations.

Preferred embodiments of this aspect of the invention employ a separation medium selected from the group consisting of beads, capillary channels and monolith structure. In particularly preferred embodiments the fixed unit of separation medium comprise a fixed bed of separation medium particles, especially particles selected from the group consisting of organic polymer and inorganic particles having a nonpolar surface.

In other embodiments of this aspect of the invention, the device for purifying a target polynucleotide has a closed lower chamber and/or the the lower chamber has an open bottom portion. The device can include an eluant container shaped to receive said lower chamber. In a particularly preferred embodiment of the invention the eluant chamber is a centrifuge vial.

In another preferred embodiment of the invention the afore-mentioned cylinder is a member of an array of cylinders and the eluant container is a member of an array of eluant containers, and the array of cylinders and array of containers have matching configurations.

In another aspect, the invention provides a separation system comprising a multicavity separation plate having outer sealing edges, a multiwell collection plate and a vacuum system having a separation plate sealing means forming a sealed engagement with the outer sealing edges of the multicavity separation plate and a vacuum cavity receiving the multiwell collection plate; the multicavity separation plate including an array of tubes, each tube having an upper solution input chamber, a lower eluant receiving chamber with an bottom opening therein, and a fixed unit of separation medium supported therein, the separation medium having nonpolar separation surfaces that are free from multivalent cations that are capable of interfering with polynucleotide separations; the multiwell collection plate having collection wells which are positioned to receive liquid from the bottom opening of the lower eluant receiving chamber.

Preferred embodiments of this aspect of the invention employ a separation medium selected from the group consisting of beads, capillary channels and monolith structure. In particularly preferred embodiments the fixed unit of separation medium comprise a fixed bed of separation medium particles, especially particles selected from the group consisting of organic polymer and inorganic particles having a nonpolar surface.

### BRIEF DESCRIPTION OF THE DRAWINGS

FIG. 1 is a cross-sectional representation of a spin vial system for low pressure separations according to this invention.

FIG. 2 is a multiwell plate separation system of this invention in combination with a vacuum attachment.

FIG. 3 is the top view of a multiwell plate of FIG. 2.

FIG. 4 is a cross-sectional view of the separation tray of FIG. 2 taken along the line A-A.

FIG. 5 is an enlarged view of a single separation cell of the multiwell plate of FIG. 4.

FIG. 6 is a chromatogram from a MIPC analysis of RNA size markers. Peaks are labeled with the number of nucleotides of the eluted molecules.

FIG. 7 is a chromatogram from a MIPC analysis of RNA size markers.

FIG. 8 is a chromatogram from a MIPC analysis of total RNA from a plant extract.

FIG. 9 is a chromatogram from a MIPC analysis of RNA from a plant extract after a first affinity purification.

FIG. 10 is a chromatogram from a MIPC analysis of RNA from a plant extract after a second affinity purification.

FIG. 11 is a chromatogram from a MIPC analysis of mouse brain mRNA.

FIG. 12 is a chromatogram from a MIPC analysis of human brain mRNA.

FIG. 13 is a chromatogram from a MIPC analysis of human brain mRNA.

FIG. 14 shows the release of eight DNA fragments from polymer beads in single equilibria bulk separations (under conditions as described in TABLE 1) showing the dependence on the acetonitrile concentration.

FIG. 15 is a separation of pUC18-DNA HaeIII digest on two discs containing binding media placed in series and containing nonporous poly(styrene-divinylbenzene) polymer beads. The dimensions of each disc was 0.7 mm×4.6 mm i.d.

FIG. 16 is a is a chromatogram of a pUC 18 Msp I standard mixture of dsDNA fragments used in Example 14.

FIG. 17 is a chromatogram of the low molecular weight and small base-pair length fraction eluant obtained in Example 14.

FIG. 18 is a chromatogram of the high base-pair length fraction eluant obtained in Example 14, demonstrating the efficacy of the spin column device for purifying high base-pair length components of a mixture of DNA fragments.

FIG. 19 is a chromatogram of a pBR322 standard mixture of dsDNA fragments used in Example 15.

FIG. 20 is a chromatogram of the low molecular weight and small base-pair length fraction eluant obtained in Example 15.

FIG. 21 is a chromatogram of the high base-pair length fraction eluant obtained in Example 15.

FIG. 22 is a chromatogram obtained in the procedure of Example 16.

FIG. 23 is a chromatogram obtained in the procedure of Example 16.

FIG. 24 shows a chromatogram obtained for unpurified product of polynucleotide kinase reaction.

FIG. 25 shows a chromatogram obtained for product of polynucleotide kinase reaction subsequent to spin column purification.

FIG. 26a is a chromatogram representing the IP-RP-HPLC separation of the FAM-labeled HJ3 strand cleaved by hydroxyl radical treatment in the absence of DNA binding protein, as described in Example 18.

FIG. 26b is a chromatogram representing the IP-RP-HPLC separation of the FAM-labeled HJ3 strand cleaved by hydroxyl radical treatment in the presence of the DNA binding protein RuvA, as described in Example 18.

FIG. 26c is a chromatogram representing the IP-RP-HPLC separation of the FAM-labeled HJ3 strand cleaved by a G+A Maxam-Gilbert sequencing reaction, generated to phase the DNA footprinting chromatograms of FIGS. 26a and 26b, as described in Example 18.

FIG. 27a is a chromatogram representing the IP-RP-HPLC separation of the TET-labeled HJ4 strand cleaved by hydroxyl radical treatment in the absence of DNA binding protein, as described in Example 18.

FIG. 27b is a chromatogram representing the IP-RP-HPLC separation of the TET-labeled HJ4 strand cleaved by hydroxyl radical treatment in the presence of the DNA binding protein RuvA, as described in Example 18.

### DETAILED DESCRIPTION OF THE INVENTION

Ion pairing reverse phase HPLC (IP-RP-HPLC) is a technique for the separation and analysis of polynucleotides that has been shown to achieve high resolution separations in a reproducible manner. IP-RP-HPLC is characterized by the use of a reverse phase (i.e., hydrophobic) stationary phase and a mobile phase that includes an alkylated cation (e.g., triethylammonium) that is believed to form a bridging interaction between the negatively charged polynucleotide and non-polar stationary phase. The alkylated cation-mediated interaction of polynucleotide and stationary phase can be modulated by the polarity of the mobile phase, conveniently adjusted by means of a solvent that is less polar than water, e.g., acetonitrile. Performance is enhanced by the use of a non-porous separation medium, as described in U.S. patent application No. 5,585,236, incorporated by reference herein in its entirety. It has been shown, for example, that under non-denaturing conditions the retention time of a double-stranded DNA fragment is dictated by the size of the fragment; the base composition or sequence of the fragment does not appreciably affect the separation, see U.S. patent application No. 5,772,889. Reproducible size-based separations of single-stranded DNA and RNA have also been achieved, see for example U.S. patent application No. 09/557,424, incorporated by reference herein in its entirety. A superior form of IP-RP-HPLC, termed Matched Ion Polynucleotide Chromatography (MIPC), is described in U.S. Pat. Nos. 5,585,236, 6,066,258 and 6,056,877 and PCT Publication Nos. WO98/48913, WO98/48914, WO/9856797, WO98/56798, incorporated herein by reference in their entirety. MIPC is characterized by the use of solvents and chromatographic surfaces that are substantially free of multivalent cation contamination that can interfere with polynucleotide separation.

Although IP-RP-HPLC is able to rapidly achieve good polynucleotide separations, the columns and other components of the system are relatively expensive. This can limit the application of the techniques for the use in the routine processing a large number of samples. It would be desirable to have available less expensive purification methods and apparatus that at least to some extent achieve the superior performance of IP-RP-HPLC, but in a more affordable format suited to the economical and rapid preparation of multiple polynucleotide. The instant invention achieves this aim, thus providing a valuable contribution to related fields of endeavour such as molecular biology and medicine.

For the sake of simplifying the explanation and not by way of limitation, the following discussion will at times reference a particular species of polynucleotide (e.g., double-stranded DNA, RNA). Nevertheless, it is to be understood that the instant invention pertains to the processing of polynucleotides in general, and is not intended to be limited to any particular species.

The present invention provides novel methods and apparatus for separating and purifying polynucleotides. This process exploits the ability of polynucleotides, in the presence of certain counterions, to bind non-specifically and reversibly to a solid phase separation medium having a hydrophobic surface, e.g., chromatography beads. In the process of the invention, the polynucleotide can be present in solution with water or in a reaction buffer. Such a solution can also contain other components, such as other biomolecules, inorganic compounds and organic compounds as long as such other components do not interfere significantly with the binding process of the invention. As an example, the solution can be a preparation of total RNA and/or genomic DNA from a cell type or organism. The process can be applied with any system which can retain the separation medium and provides means to rapidly pass liquids through the separation medium.

A similar process, IP-RP-HPLC (discussed above) has been shown to be effective for separating polynucleotides. The instant invention pertains to the use of non-HPLC chromatographic methods for separating and/or purifying polynucleotides. The term “non-HPLC chromatographic method” is intended to encompass any chromatographic method which does not involve the use of a pump to generate high pressure to force eluant through a chromatography column. HPLC separations are typically achieved at pressures greater than 1000 psi, often reaching 2000 to 3000 psi, and in some instances reaching 6000 psi or higher. The requirements for an HPLC pumping system are severe and normally include some or all of the following features: (1) the generation of pressures of up to 6000 psi or more, (2) a substantially pulse-free output, (3) flow rates ranging from 0.1 to 10 mL/min, (4) flow control and flow reproducibility of 0.5% relative or better, and (5) corrosion-resistant components (seals of stainless steel or Teflon). Non-HPLC chromatographic methods are characterized by the use of alternate means for driving the eluant through the column. Non-limiting examples of such alternate means include gravity, low or medium pressure pumps (e.g., peristaltic pumps), centrifugal force, high pressure gas and vacuum pressure. In general, non-HPLC chromatography methods are more economical than HPLC, which represents a significant advantage of the instant invention.

In a preferred embodiment of the invention, the method is able to isolate a target polynucleotide, or target polynucleotides sharing predetermined physical characteristics (e.g., size ranges, hydrophobicity, poly-A tails, etc.), from a larger pool of non-target molecules (e.g., biomolecules, non-target polynucleotides). Exemplary applications of the invention include purification of plasmid DNA (e.g., from a miniprep), purification of the product of PCR amplification, purification of chemically synthesized oligonucleotide, and recovery of polynucleotide after enzymatic modification (e.g., phosphorylation by polynucleotide kinase). The invention can be used to isolate a pool of RNAs enriched for a particular class of RNA molecules (e.g., mRNAs, rRNAs, tRNAs). The method can also be used to separate selected RNA molecules from other macromolecules (e.g., genomic DNA, proteins, carbohydrates) or small molecule contaminants. In a particularly preferred embodiment of the invention, the method can be used to stabilize RNA molecules by separating the RNA from species capable of promoting RNA degradation, particularly RNases and other nucleases.

One of the advantages of the instant invention that distinguish it over previously available separation procedures is the ability to effectively and predictably separate long polynucleotides. Hence, in preferred embodiments of the invention the method is used to separated polynucleotides (double- or single-stranded) of a length greater than 100 nucleotides, more preferably greater than 500 nucleotides, still more preferably longer than 1000 nucleotides, even more preferably longer than 1500 nucleotides, and most preferably greater than 2500 nucleotides.

The invention is particularly useful for the separation of tagged polynucleotides. Non-limiting examples of polynucleotides tags suitable for use with the instant invention include fluorescent groups, hydrophobic or hydrophillic groupls, biotin, digoxigenin, etc.. Non-limiting examples of fluorescent groups suitable for use with the instant invention include 5-carboxyfluorescein (5-FAM), 6-carboxyfluorescein (6-FAM), 2′,7′-dimethoxy-4′,5′-dichloro-6-carboxyfluorescein (JOE), N,N,N′-N-tetramethyl-6-carboxy rhodamine (TAMRA), 6-carboxy-X-rhodamine (ROX), 4,7,2′,4′,5′,7′-hexachloro-6-carboxy-fluorescein (HEX-1), 4,7,2′,4′,5′,7′-hexachloro-5-carboxy-fluorescein (HEX-2), 2′,4′,5′,7′-tetrachloro-5-carboxy-fluorescein (ZOE), 4,7,2′,7′-tetrachloro-6-carboxy-fluorescein (TET-1), 1′,2′,7′,8′-dibenzo-4,7-dichloro-5-carboxyfluorescein (NAN-2), and 1′,2′,7′,8′-dibenzo-4,7-dichloro-6-carboxyfluorescein, fluorescein and fluorescein derivatives, Rhodamine, Cascade Blue, Alexa₃₅₀, Alexa₄₈₈, , phycoerythrin, allo-phycocyanin, phycocyanin, rhodamine, Texas Red, EDANS, BODIPY dyes such as BODIPY-FL and BODIPY-TR-X, tetramethylrhodamine, Cy3 and Cy5, 5,6-carboxyfluorescein, fluorescein mono-derivatized with a linking functionality at either the 5 or 6 carbon position, including fluorescein-5-isothiocyanate, fluorescein-6-isothiocyanate (the −5- and −6-forms being referred to collectively as FITC), fluorescein-5-succinimidylcarboxylate, fluorescein-6-succinimidylcarboxylate, fluorescein-5-iodoacetamide, fluorescein-6-iodoacetamide, fluorescein-5-maleimide, and fluorescein-6-maleimide; , 2′,7′-dimethoxy-4′,5′-dichlorofluorescein mono-derivatized with a linking functionality at the 5 or 6 carbon position, including 2′,7′-dimethoxy-4′,5′-dichlorofluorescein-5-succinimidylcarboxylate and 2,′,7′-dimethoxy-4′,5′-dichlorofluoescein-6-succinimidylcarboxylate (the −5- and −6-forms being referred to collectively as DDFCS), tetramethylrhodamine mono-derivatized with a linking functionality at either the 5 or 6 carbon position, including tetramethylrhodamine-5-isothiocyanate, tetramethylrhodamine-6-isothiocyanate (the −5- and −6-forms being referred to collectively as TMRITC), tetramethylrhodamine-5-iodoacetamide, tetramethylrhodamine-6-iodoacetamide, tetramethylrhodamine-5-succinimidylcarboxylate, tetramethylrhodamine-6-succinimidylcarboxylate, tetramethylrhodamine-5-maleimide, and tetramethylrhodamine-6-maleimide, rhodamine X derivatives having a disubstituted phenyl attached to the molecule's oxygen heterocycle, one of the substituents being a linking functionality attached to the 4′ or 5′ carbon (IUPAC numbering) of the phenyl, and the other being a acidic anionic group attached to the 2′ carbon, including Texas Red (tradename of Molecular Probes, Inc.), rhodamine X-5-isothiocyanate, rhodamine X-6-isothiocyanate, rhodamine X-5-iodoacetamide, rhodamine X-6-iodoacetamide, rhodamine X-5-succinimidylcarboxylate, rhodamine X-6-succinimidylcarboxylate, rhodamine X-5-maleimide, and rhodamine X-6-maleimide.

Fluorescent labels can be attached to DNA using standard procedures, e.g. for a review see Haugland, “Covalent Fluorescent Probes,” in Excited States of Biopolymers, Steiner, Ed. (Plenum Press, New York, 1983), incorporated by reference herein in its entirety. In a preferred embodiment of the invention, a fluorescent group can be covalently attached to a desired primer by reaction with a 5′-amino-modified oligonucleotide in the presence of sodium bicarbonate and dimethylformamide, as described in U.S. patent application No. 09/169,440. Alternatively, the reactive amine can be attached by means of the linking agents disclosed in U.S. Pat. No. 4,757,141. Alternatively, covalently tagged primers can be obtained commercially (e.g., from Midland Certified Reagent, Co.). Fluorescent dyes are available form Molecular Probes, Inc. (Eugene, Oreg.), Operon Technologies, Inc., (Alameda, Calif.) and Amersham Pharmacia Biotech (Piscataway, N.J.), or can be synthesized using standard techniques. Fluorescent labeling is described in U.S. Pat. No. 4,855,225.

Polynucleotides for use in the disclosed method can be part of a crude cellular or nuclear extract, partially purified, or extensively purified. DNA molecules can be the product of in vivo or in vitro amplification (e.g., PCR) or chemical synthesis (oligonucleotides). RNA molecules can also be made by in vitro transcription or by direct synthesis. The method can be used, for example, to purify an individual polynucleotide or a plurality of polynucleotides (e.g., a synthetic oligonucleotide or PCR amplification product), to separate polynucleotides from other biomolecules, to separate one species of polyucleotide from another (e.g., genomic DNA from RNA or plasmid DNA, mRNA from other RNA species). Polynucleotides can be prepared using known methods for preparing cellular extracts and for purifying polynucleotides. Methods for preparing extracts containing DNA and/or RNA molecules are described in, for example, Sambrook et al., and Ausubel et al. Individual DNA molecules can also be produced recombinantly using known techniques, by in vitro transcription, and by direct synthesis. For recombinant and in vitro transcription, DNA encoding RNA molecules can be obtained from known clones, by synthesizing a DNA molecule encoding an RNA molecule, or by cloning the gene encoding the RNA molecules. Techniques for in vitro transcription of RNA molecules and methods for cloning genes encoding known RNA molecules are described by, for example, Sambrook et al. Polynucleotides can be prepared, for example, on an Applied Biosystems (Foster City, Calif.) 392 DNA/RNA synthesizer using standard phosporamidite chemistry.

The method can be applied to an RNA preparation that has been enriched for RNA containing poly-A tails (associated with most mature mRNA molecules) from total RNA. These can be prepared by affinity chromatography using beads coated with poly-T oligomers, as described, for example, in Sambrook and Ausubel. Separation columns containing such beads are commercially available from a number of sources (e.g., Poly(A)Pure™ mRNA Purification Kit, Ambion, Inc., Austin, Tex.; Oligotex™ mRNA Purification System, Qiagen, Inc., Valencia, Calif.).

In a representative general embodiment of the invention provided for purposes of illustration, a first solution containing a polynucleotide, or a collection of polynucleotides, is applied to a separation medium having a nonpolar, preferably nonporous surface, the first solution containing counterion and a polynucleotide-binding concentration of organic solvent, whereby a target polynucleotide, or plurality of polynucleotides, is non-specifically and reversibly bound to the medium. The target polynucleotide or polynucleotides are then removed from the medium by contacting the medium with a second solution containing counterion and a concentration of organic solvent sufficient to elute the target polynucleotide or polynucleotides from the separation medium into a distinct segment of eluant. In a preferred embodiment, the concentration of organic solvent sufficient to elute the target polynucleotides is predetermined based on the length and/or physical characteristics of the target.

In one embodiment of the process of the present invention, the separation can be conducted as a batch process in a container. The volume of the container can vary widely depending on the amount of mixture to be separated. The container can be, for example, a low-pressure (e.g., ambient pressure) column, a spin column, a web, a pad, a flask, a well, or a tank. The size of such a container can be as small as a well on a multi-well microtiter plate or as large as a multi-liter vat, for example. In a preferred embodiment the separation medium takes the form of chromatographic beads. Beads useful in the batch process can be a variety of shapes, which can be regular or irregular; preferably the shape maximizes the surface area of the beads. The beads should be of such a size that their separation from solution, for example by filtration or centrifugation, is not difficult.

The term “polynucleotide” is defined as a polymer containing an indefinite number of nucleotides, linked from one ribose (or deoxyribose) to another via phosphodiester bonds. The present invention can be used in the separation of RNA or of double- or single-stranded DNA or of synthetic nucleic acid analogs. The polynucleotide can be a linear molecule or a closed circle and can be modified, e.g. labeled with biotin or fluorescent molecules. For purposes of simplifying the description of the invention, and not by way of limitation, the separation of a particular species of polynucleotide (e.g., dsDNA, RNA, ssDNA) will be described in the examples herein, it being understood that all polynucleotides are intended to be included within the scope of this invention. Short, typically single-stranded polynucleotides are referred to as oligonucleotides, and are often used in molecular biology as primers and probes.

The term “counterion agent” is defined herein as a compound used to form a ionic pair with a polynucleotide that is capable of separation by the methods described herein. Preferred counterion agents comprise a cationic species having a hydrophobic character (e.g., an alkylated cation such as triethylammonium), believed to be capable of forming a bridging interaction between negatively charged polynucleotides and the hydrophobic surface of a separation medium of the invention.

“Non-specific binding” refers to the binding of a plurality of polynucleotides in a mixture despite differences in the sequence or size of the different polynucleotides. In the present invention, such binding occurs when the fragments are exposed to the hydrophobic surface of a separation medium in a solvent containing a suitable counterion agent but lacking a sufficient concentration of organic solvent to cause release of the bound polynucleotides.

“Separation medium” refers to a solid phase having a hydrophobic surface suitable for binding polynucleotides in the presence of an aqueous phase containing a suitable counterion agent. Examples include beads, particles and monoliths.

“Elution solution” refers to an aqueous solution containing a concentration of organic solvent sufficient to cause the elution of a polynucleotide, especially a target polynucleotide, from the hydrophobic surface of the separation medium. The concentration of organic solvent need not be sufficient to result in the elution of all polynucleotide species, e.g., non-target polynucleotides.

The term “organic solvent” refers to a solvent of sufficient non-polar character to cause elution of a polynucleotide from a separation medium when used as a component of an elution solution. Preparation of elution solution is facilitated by the used of an organic solvent that is suitably water-soluble.

The term “loading solution” refers to a solution containing a target polynucleotide that is applied to a separation medium for purification according to the present invention. In preferred embodiments of the invention the loading solution is aqueous and includes a counterion agent.

The term “purify” is used in the present invention to describe the separation of a target polynucleotide from some other molecular constituent of the loading solution, i.e., a non-target molecule, such as a different polynucleotide or other biomolecule. The term purify does not necessarily imply a total separation from all other polynucleotides or molecular species. For example, in some embodiments of the invention a family of related polynucleotides (e.g., mRNAs) is separated from another class of polynucleotide (e.g., genomic DNA).

The term “wash solution” refers to a solution used to wash non-target molecule from the separation medium with no substantial release of target polynucleotide. A wash solution will generally contain a concentration of organic solvent sufficient to elute non-target molecule, but insufficient to elute target polynucleotide. In preferred embodiments of the invention the wash solution contains a counterion agent.

The apparatus of this invention provides a novel and unique method for separating and purifying single-stranded oligonucleotides and single-stranded DNA fragments, RNA, double-stranded DNA fragments, plasmids and the like. This process exploits the binding characteristics of separation media with nonpolar surfaces in the presence of counterion and materials to be separated. Materials in aqueous solutions of the counterion and low organic solvent concentrations bind to the nonpolar surfaces, and the materials are subsequently released from the surface by application of an elution solution of sufficient non-polar character to cause the elution of target polynucleotides. In general, the concentration of organic solvent required to cause elution increases with increasing length of the target polynucleotide. In many cases, the ratio of fragment size desorbed from the media to the concentration of organic solvent can be calibrated and is so reproducible that it can be calculated with high accuracy. The process can be applied with any system which can retain the separation medium and provides means to rapidly pass liquids through the separation medium.

Practice of the instant invention can entail a variety of techniques and methods known to one of skill in the art. Such methods are widely available and provided, for example, in Molecular Cloning: a Laboratory Manual: 2nd edition, 3 Volumes, Sambrook et al, 1989, Cold Spring Harbor Laboratory Press (or later editions of the same work) or Current Protocols in Molecular Biology, Second Edition, Ausubel et al. eds., John Wiley & Sons, 1992.

The separation medium is a unique aspect of this invention. In general, the separation medium should have a surface that is either intrinsically non-polar or bonded with a material that forms a surface having sufficient non-polarity to interact with a counterion agent. In a preferred embodiment the medium takes the form of chromatographic beads. The media surfaces can be porous or nonporous. Examples of porous media are described in U.S. Pat. No. 5,972,222. Examples of the preferred nonporous media are described in U.S. Pat. Nos. 5,585,236, 6,066,258 and 6,056,877. In a preferred embodiment of the invention, the surfaces of the medium should be free of any traces of metal contaminants, particularly multivalent metal ions.

To effect rapid and precise separations, nonporous media surfaces are preferred, i.e., beads having a pore size that essentially excludes the polynucleotides being separated from entering the bead, although porous beads can also be used. As used herein, the term “nonporous” is defined to denote a bead that has surface pores having a diameter that is sufficiently small so as to effectively exclude the smallest RNA fragment in the separation in the solvent medium used therein. Included in this definition are polymer beads having these specified maximum size restrictions in their natural state or which have been treated to reduce their pore size to meet the maximum effective pore size required.

The surface conformations of nonporous beads of the present invention can include depressions and shallow pit-like structures that do not interfere with the separation process. A pretreatment of a porous bead to render it nonporous can be effected with any material which will fill the pores in the bead structure and which does not significantly interfere with the MIPC process.

Non-porous polymeric beads useful in the practice of the present invention can be prepared by a two-step process in which small seed beads are initially produced by emulsion polymerization of suitable polymerizable monomers. The emulsion polymerization procedure is a modification of the procedure of Goodwin, et al. (Colloid & Polymer Sci., 252:464-471 (1974)). Monomers which can be used in the emulsion polymerization process to produce the seed beads include styrene, alkyl substituted styrenes, alpha-methyl styrene, and alkyl substituted alpha-methyl styrene. The seed beads are then enlarged and, optionally, modified by substitution with various groups to produce the nonporous polymeric beads of the present invention.

The seed beads produced by emulsion polymerization can be enlarged by any known process for increasing the size of the polymer beads. For example, polymer beads can be enlarged by the activated swelling process disclosed in U.S. Pat. No. 4,563,510. The enlarged or swollen polymer beads are further swollen with a crosslinking polymerizable monomer and a polymerization initiator. Polymerization increases the crosslinking density of the enlarged polymeric bead and reduces the surface porosity of the bead. Suitable crosslinking monomers contain at least two carbon-carbon double bonds capable of polymerization in the presence of an initiator. Preferred crosslinking monomers are divinyl monomers, preferably alkyl and aryl (phenyl, naphthyl, etc.) divinyl monomers and include divinyl benzene, butadiene, etc. Activated swelling of the polymeric seed beads is useful to produce polymer beads having an average diameter ranging from 1 up to about 100 microns.

Alternatively, the polymer seed beads can be enlarged simply by heating the seed latex resulting from emulsion polymerization. This alternative eliminates the need for activated swelling of the seed beads with an activating solvent. Instead, the seed latex is mixed with the crosslinking monomer and polymerization initiator described above, together with or without a water-miscible solvent for the crosslinking monomer. Suitable solvents include acetone, tetrahydrofuran (THF), methanol, and dioxane. The resulting mixture is heated for about 1-12 hours, preferably about 4-8 hours, at a temperature below the initiation temperature of the polymerization initiator, generally, about 10° C.-80° C., preferably 30° C.-60° C. Optionally, the temperature of the mixture can be increased by 10-20% and the mixture heated for an additional 1 to 4 hours. The ratio of monomer to polymerization initiator is at least 100:1, preferably in the range of about 100:1 to about 500:1, more preferably about 200:1 in order to ensure a degree of polymerization of at least 200. Beads having this degree of polymerization are sufficiently pressure-stable to be used in HPLC applications. This thermal swelling process allows one to increase the size of the bead by about 110-160% to obtain polymer beads having an average diameter up to about 5 microns, preferably about 2-3 microns. The thermal swelling procedure can, therefore, be used to produce smaller particle sizes previously accessible only by the activated swelling procedure.

Following thermal enlargement, excess crosslinking monomer is removed and the particles are polymerized by exposure to ultraviolet light or heat. Polymerization can be conducted, for example, by heating of the enlarged particles to the activation temperature of the polymerization initiator and continuing polymerization until the desired degree of polymerization has been achieved. Continued heating and polymerization allows one to obtain beads having a degree of polymerization greater than 500.

For use in the present invention, packing material disclosed by U.S. Pat. No. 4,563,510 can be modified through substitution of the polymeric beads with alkyl groups or can be used in its unmodified state. For example, the polymer beads can be alkylated with 1 or 2 carbon atoms by contacting the beads with an alkylating agent, such as methyl iodide or ethyl iodide. Alkylation can be achieved by mixing the polymer beads with the alkyl halide in the presence of a Friedel-Crafts catalyst to effect electrophilic aromatic substitution on the aromatic rings at the surface of the polymer blend. Suitable Friedel-Crafts catalysts are well-known in the art and include Lewis acids such as aluminum chloride, boron trifluoride, tin tetrachloride, etc. The beads can be hydrocarbon substituted by substituting the corresponding hydrocarbon halide for methyl iodide in the above procedure, for example.

The term alkyl as used herein in reference to the beads useful in the practice of the present invention is defined to include alkyl and alkyl substituted aryl groups, having from 1 to 1,000,000 carbons, the alkyl groups including straight chained, branch chained, cyclic, saturated, unsaturated nonionic functional groups of various types including aldehyde, ketone, ester, ether, alkyl groups, and the like, and the aryl groups including as monocyclic, bicyclic, and tricyclic aromatic hydrocarbon groups including phenyl, naphthyl, and the like. Methods for alkyl substitution are conventional and well-known in the art and are not an aspect of this invention. The substitution can also contain hydroxy, cyano, nitro groups, or the like which are considered to be non-polar, reverse phase functional groups.

Non-limiting examples of base polymers suitable for use in producing such polymer beads include mono- and di-vinyl substituted aromatics such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(styrene-divinylbenzene) and poly(ethylvinylbenzene-divinylbenzene). Methods for making beads from these polymers are conventional and well known in the art (for example, see U.S. Pat. No. 4,906,378). The physical properties of the surface and near-surface areas of the beads are the primary determinant of chromatographic efficiency. The polymer, whether derivatized or not, should provide a nonporous, non-reactive, and non-polar surface for the IP-RP-HPLC separation. In a particularly preferred embodiment of the invention, the separation medium consists of octadecyl modified, nonporous alkylated poly(styrene-divinylbenzene) beads. Separation columns employing these particularly preferred beads, referred to as DNASep® columns, are commercially available from Transgenomic, Inc.

A separation bead used in the invention can comprise a nonporous particle which has non-polar molecules or a non-polar polymer attached to or coated on its surface. In general, such beads comprise nonporous particles which have been coated with a polymer or which have substantially all surface substrate groups reacted with a non-polar hydrocarbon or substituted hydrocarbon group, and any remaining surface substrate groups endcapped with a tri(lower alkyl)chlorosilane or tetra(lower alkyl)dichlorodisilazane as described in U.S. Pat. No. 6,056,877.

The nonporous particle is preferably an inorganic particle, but can be a nonporous organic particle. The nonporous particle can be, for example, silica, silica carbide, silica nitrite, titanium oxide, aluminum oxide, zirconium oxide, carbon, insoluble polysaccharides such as cellulose, or diatomaceous earth, or any of these materials which have been modified to be nonporous. Examples of carbon particles include diamond and graphite which have been treated to remove any interfering contaminants. The preferred particles are essentially non-deformable and can withstand high pressures. The nonporous particle is prepared by known procedures. The preferred particle size is about 0.5-100 microns; preferably, 1-10 microns; more preferably, 1-5 microns. Beads having an average diameter of 1.0-3.0 microns are most preferred.

An inorganic particle must have a hydrophobic surface to function as a separation medium in the instant invention. The hydrophobic surface can be an organic polymer supported on the inorganic particle. In one embodiment, the hydrophobic surface includes long chain hydrocarbons having from 1-24 carbons, and preferably 8-24 cabons, bonded to the inorganic oxide particle. An example is a silica particle having substantially all surface substrate groups reacted with a hydrocarbon group and then endcapped with a non-polar hydrocarbon or substituted hydrocarbon group, preferably a tri(lower alkyl)chlorosilane or tetra(lower alkyl)dichlorodisilazane. The particle can be end-capped with trimethylsilyl chloride or hexamethyidisilazane.

Because the chemistry of preparing conventional silica-based reverse phase HPLC materials is well-known, most of the description of non-porous beads suitable for use in the instant invention is presented in reference to silica. It is to be understood, however, that other nonporous particles, such as those listed above, can be modified in the same manner and substituted for silica. For a description of the general chemistry of silica, see Poole, Colin F. and Salwa K. Poole, Chromatography Today, Elsevier:New York (1991), pp. 313-342 and Snyder, R. L. and J. J. Kirkland, Introduction to Modern Liquid Chromatography, 2^{nd} ed., John Wiley & Sons, Inc.:New York (1979), pp. 272-278, the disclosures of which are hereby incorporated herein by reference in their entireties.

The nonporous beads of the invention are characterized by having minimum exposed silanol groups after reaction with the coating or silating reagents. Minimum silanol groups are needed to reduce the interaction of the RNA with the substrate and also to improve the stability of the material in a high pH and aqueous environment. Silanol groups can be harmful because they can repel the negative charge of the RNA molecule, preventing or limiting the interaction of the RNA with the stationary phase of the column. Another possible mechanism of interaction is that the silanol can act as ion exchange sites, taking up metals such as iron (III) or chromium (III). Iron (III) or other metals which are trapped on the column can distort the RNA peaks or even prevent RNA from being eluted from the column.

Silanol groups can be hydrolyzed by the aqueous-based mobile phase. Hydrolysis will increase the polarity and reactivity of the stationary phase by exposing more silanol sites, or by exposing metals that can be present in the silica core. Hydrolysis will be more prevalent with increased underivatized silanol groups. The effect of silanol groups on the RNA separation depends on which mechanism of interference is most prevalent. For example, iron (III) can become attached to the exposed silanol sites, depending on whether the iron (III) is present in the eluent, instrument or sample.

The effect of metals can occur if metals are present within the system or reagents. Metals present within the system or reagents can get trapped by ion exchange sites on the silica. However, if no metals are present within the system or reagents, then the silanol groups themselves can cause interference with RNA separations. Hydrolysis of the exposed silanol sites by the aqueous environment can expose metals that might be present in the silica core.

Fully hydrolyzed silica contains a concentration of about 8 μmoles of silanol groups per square meter of surface. At best, because of steric considerations, a maximum of about 4.5 μmoles of silanol groups per square meter can be reacted, the remainder of the silanol being sterically shielded by the reacted groups. Minimum silanol groups is defined as reaching the theoretical limit of or having sufficient shield to prevent silanol groups from interfering with the separation.

Numerous methods exist for forming nonporous silica core particles. For example, sodium silicate solution poured into methanol will produce a suspension of finely divided spherical particles of sodium silicate. These particles are neutralized by reaction with acid. In this way, globular particles of silica gel are obtained having a diameter of about 1-2 microns. Silica can be precipitated from organic liquids or from a vapor. At high temperature (about 2000° C.), silica is vaporized, and the vapors can be condensed to form finely divided silica either by a reduction in temperature or by using an oxidizing gas. The synthesis and properties of silica are described by R. K. Iler in The Chemistry of Silica, Solubility, Polymerization, Colloid and Surface Properties, and Biochemistry, John Wiley & Sons:New York (1979).

W. Stöber et al. described controlled growth of monodisperse silica spheres in the micron size range in J. Colloid and Interface Sci., 26:62-69 (1968). Stöber et al. describe a system of chemical reactions which permit the controlled growth of spherical silica particles of uniform size by means of hydrolysis of alkyl silicates and subsequent condensation of silicic acid in alcoholic solutions. Ammonia is used as a morphological catalyst. Particle sizes obtained in suspension range from less than 0.05 μm to 2 μm in diameter.

To prepare a nonporous bead, the nonporous particle can be coated with a polymer or reacted and endcapped so that substantially all surface substrate groups of the nonporous particle are blocked with a non-polar hydrocarbon or substituted hydrocarbon group. This can be accomplished by any of several methods described in U.S. Pat. No. 6,056,877. Care should be taken during the preparation of the beads to ensure that the surface of the beads has minimum silanol or metal oxide exposure and that the surface remains nonporous. Nonporous silica core beads can be obtained from Micra Scientific (Northbrook, Ill.) and from Chemie Uetikkon (Lausanne, Switzerland).

Beads useful in the present process can be a variety of shapes, which can be regular or irregular; preferably the shape maximizes the surface area of the beads. The beads should be of a size such that their separation from solution, for example by filtration or centrifugation, is not difficult.

In another embodiment of the present invention, the separation medium can be in the form of a polymeric monolith, e.g., a rod-like monolithic column. A monolith is a polymer separation medium, formed inside a column, having a unitary structure with through pores or interstitial spaces that allow eluting solvent and analyte to pass through and which provide the non-polar separation surface, as described in U.S. Pat. No. 6,066,258 and U.S. patent application No. 09/562,069. The interstitial separation surfaces can be porous, but are preferably nonporous. The separation principles involved parallel those encountered with bead-packed columns. As with beads, pores traversing the monolith must be compatible with and permeable to polynculeotides. In a preferred embodiment, the rod is substantially free of contamination capable of reacting with polynucleotides and interfering with its separation, e.g., multivalent cations.

A molded polymeric monolith rod that can be used in practicing the present invention can be prepared, for example, by bulk free radical polymerization within the confines of a chromatographic column. The base polymer of the rod can be produced from a variety of polymerizable monomers. For example, the monolithic rod can be made from polymers, including mono- and di-vinyl substituted aromatic compounds such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(glycidyl methacrylate-co-ethylene dimethacrylate), poly(styrene-divinylbenzene) and poly(ethylvinylbenzene-divinylbenzene. The rod can be unsubsituted or substituted with a substituent such as a hydrocarbon alkyl or an aryl group. The alkyl group optionally has 1 to 1,000,000 carbons inclusive in a straight or branched chain, and includes straight chained, branch chained, cyclic, saturated, unsaturated nonionic functional groups of various types including aldehyde, ketone, ester, ether, alkyl groups, and the like, and the aryl groups includes as monocyclic, bicyclic, and tricyclic aromatic hydrocarbon groups including phenyl, naphthyl, and the like. In a preferred embodiment, the alkyl group has 1-24 carbons. In a more preferred embodiment, the alkyl group has 1-8 carbons. The substitution can also contain hydroxy, cyano, nitro groups, or the like which are considered to be non-polar, reverse phase functional groups. Methods for hydrocarbon substitution are conventional and well-known in the art and are not an aspect of this invention. The preparation of polymeric monoliths is by conventional methods well known in the art as described in the following references: Wang et al.(1 994) J. Chromatog. A 699:230; Petro et al. (1996) Anal. Chem. 68:315 and U.S. Pat. Nos. 5,334,310; 5,453,185 and 5,522,994. Monolith or rod columns are commercially available form Merck & Co (Darmstadt, Germany).

The separation medium can take the form of a continuous monolithic silica gel. A molded monolith can be prepared by polymerization within the confines of a column (e.g., to form a rod) or other containment system. A monolith is preferably obtained by the hydrolysis and polycondensation of alkoxysilanes. A preferred monolith is derivatized in order to produce non-polar interstitial surfaces. Chemical modification of silica monoliths with ocatdecyl, methyl or other ligands can be carried out. An example of a preferred derivatized monolith is one which is polyfunctionally derivatized with octadecylsilyl groups. The preparation of derivatized silica monoliths can be accomplished using conventional methods well known in the art as described in the following references which are hereby incorporated in their entirety herein: U.S. Pat. No. 6,056,877, Nakanishi, et al., J. Sol-Gel Sci. Technol. 8:547 (1997); Nakanishi, et al., Bull, Chem. Soc. Jpn. 67:1327 (1994); Cabrera, et al., Trends Analytical Chem. 17:50 (1998); Jinno, et al., Chromatographia 27:288 (1989).

The present invention preferably employs a separation medium having low amounts of metal contaminants or other contaminants that can bind RNA. For example, preferred beads have been produced under conditions where precautions have been taken to substantially eliminate any multivalent cation contaminants (e.g. Fe(III), Cr(III), or colloidal metal contaminants), including a decontamination treatment, e.g., an acid wash treatment. Only very pure, non-metal containing materials should be used in the production of the beads in order to minimize the metal content of the resulting beads.

In addition to the separation medium being substantially metal-free, to achieve optimum peak separation all process solutions and materials contacting the medium are preferably substantially free of multivalent cation contaminants (e.g. Fe(III), Cr(III), and colloidal metal contaminants). For example, all surfaces contacting the separation medium or process solution are preferably made of material which does not release multivalent cations, as described (in the context of HPLC) in U.S. Pat. Nos. 5,772,889, 5,997,742 and 6,017,457. Preferred materials include titanium, coated stainless steel, passivated stainless steel, and organic polymer. Metals found in stainless steel, for example, do not harm the separation, unless they are in an oxidized or colloidal partially oxidized state.

For additional protection, multivalent cations in mobile phase solutions and sample solutions can be removed by contacting these solutions with a multivalent cation capture resin. The multivalent capture resin is preferably cation exchange resin and/or chelating resin. An example of a suitable chelating resin is available under the trademark CHELEX 100 (Dow Chemical Co.) containing an iminodiacetate functional group.

In another embodiment, a multivalent cation-binding agent can be added to solutions used in the invention. The multivalent cation-binding agent can be a coordination compound. Examples of preferred coordination compounds include water soluble chelating agents and crown ethers. Non-limiting examples of multivalent cation-binding agents which can be used in the present invention include acetylacetone, alizarin, aluminon, chloranilic acid, kojic acid, morin, rhodizonic acid, thionalide, thiourea, α-furildioxime, nioxime, salicylaldoxime, dimethylglyoxime, α-furildioxime, cupferron, α-nitroso-β-naphthol, nitroso-R-salt, diphenylthiocarbazone, diphenylcarbazone, eriochrome black T, PAN, SPADNS, glyoxal-bis(2-hydroxyanil), murexide, α-benzoinoxime, mandelic acid, anthranilic acid, ethylenediamine, glycine, triaminotriethylamine, thionalide, triethylenetetramine, EDTA, metalphthalein, arsonic acids, α,α′-bipyridine, 4-hydroxybenzothiazole, 8-hydroxyquinaldine, 8-hydroxyquinoline, 1,10-phenanthroline, picolinic acid, quinaldic acid, α,α′,α″-terpyridyl, 9-methyl-2,3,7-trihydroxy-6-fluorone, pyrocatechol, salicylic acid, tiron, 4-chloro-1,2-dimercaptobenzene, dithiol, mercaptobenzothiazole, rubeanic acid, oxalic acid, sodium diethyldithiocarbarbamate, and zinc dibenzyldithiocarbamate. These and other examples are described by Perrin in Organic Complexing Reagents: Structure, Behavior, and Application to Inorganic Analysis, Robert E. Krieger Publishing Co. (1964). In the present invention, a preferred multivalent cation-binding agent is EDTA.

The present invention requires a counterion agent for forming a hydrophobic salt with anionic RNA to enable the hydrophobic interaction of the RNA-counterion with the separation medium. Counterion agents that are volatile, such as trialkylammonium acetate and trialkylammonium carbonate, are preferred for use in the process of the invention, with triethylammonium acetate (TEAA) and triethylammonium hexafluoroisopropyl alcohol being most preferred. Trialkylammonium phosphate can also be used. The counterion agent can be added to the RNA preparation first, or the RNA preparation can be injected into a polar stripping solvent containing the counterion agent. Preferred counterion agents are those which are easily removed after the separation process. In that regard, volatile salts are desired because they are easily removed from the purified product by evaporation. The presence of non-volatile salts, associated with some previously available polynucleotide separation methods, can interfere with further processing and analysis of the purified polynucleotide. The ability to use non-volatile salts is a significant advantage of the instant invention.

The counterion agent is preferably selected from the group consisting of lower alkyl primary amine, lower alkyl secondary amine, lower alkyl tertiary amine, lower trialkyammonium salt, quaternary ammonium salt, and mixtures of one or more thereof. Non-limiting examples of counterion agents include octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyldiethylammonium acetate, butylethylammonium acetate, methylhexylammonium acetate, tetramethylammonium acetate, tetrapropylammonium acetate, tetrabutylammonium acetate, dimethydiethylammonium acetate, triethylammonium acetate, tripropylammonium acetate, tributylammonium acetate, tetraethylammonium acetate, tetrapropylammonium acetate, tetrabutylammonium acetate, and mixtures of any one or more of the above.

Although the anion in the above examples is acetate, other anions may also be used, including carbonate, phosphate, sulfate, nitrate, propionate, formate, chloride, and bromide, or any combination of cation and anion. These and other agents are described by Gjerde, et al. in Ion Chromatography, 2nd Ed., Dr. Alfred Hüthig Verlag Heidelberg (1987).

The pH of solutions used in the present invention, including loading solutions and stripping solvents, is preferably within the range of about pH 5 to about pH 9, and optimally within the range of about pH 6 to about pH 7.5.

In some embodiments of the invention it is desirable to perform the separation under denaturing conditions. The term “denaturing conditions” refers to conditions where polynucleotides of interest (normally mRNA molecules on the context of the instant invention) are denatured, resulting in substantial loss of secondary structure and/or tertiary structure, which can improve the separation. In particular, separation of single-stranded polynucleotides under denaturing conditions can result in enhanced size-dependency of the separation. Denaturing conditions can be achieved, for example, by conducting chromatography at high temperature (usually at about 50° C. or greater, preferably at about 50° C. or greater, and most preferably at about 75° C. or greater), at a pH sufficient to cause denaturation, in the presence of a chemical denaturant, or a combination thereof. Normally, extreme pH is not a preferred means of achieving denaturation owing to the instability of RNA under both acid and base conditions. High temperature can be achieved by heating the separation medium during separation. For example, a spin column can be centrifuged in a heated environment. Alternatively, in some cases it is more convenient to pre-heat the sample (e.g., the loading solution) prior to performing the separation under ambient temperature conditions, which will achieve satisfactory results if the time of separation (e.g., the time of the centrifugation of an elution solution through a spin column) is short enough that the denatured state of the polynucleotide is maintained. Alternatively, a multicavity separation system as described below can be heated by means of a heat block or similar structure.

Other features of the invention will become apparent in the course of the following descriptions of exemplary embodiments which are given for illustration of the invention and are not intended to be limiting thereof.

The present invention involves polynucleotide elution by means of an elution solution containing an appropriate concentration of organic solvent. At increasing organic solvent concentrations polynucleotides can be released from the separation medium as a function of physical properties that affect interaction with the medium, particularly size and hydrophobicity. Preferred organic solvents are able to release the polynucleotide-counterion complex from the separation medium surface while maintaining the complex in solution. Preferred organic solvents do not interfere with the isolation or recovery of the fragments and are easily removed after the separation. Examples of suitable organic solvents include alcohol, nitrile, dimethylformamide, tetrahydrofuran, ester, ether, and mixtures of one or more thereof, e.g., methanol, ethanol, 2-propanol, 1-propanol, tetrahydrofuran, ethyl acetate, acetonitrile. The most preferred organic solvent is acetonitrile. The presence of concentrations of acetonitrile sufficient to elute a polynucleotide according to the instant invention have been shown not to inhibit various enzymes used in molecular biology protocols, such as polymerases and restriction enzymes. Thus, in many cases a polynucleotide purified according to the instant invention can be used directly in downstream applications (e.g. RT-PCR, sequencing), without removal of the acetonitrile.

In the process of the present invention, the release of the fragments from the surface can be modulated by exposing the surface of the separation medium to variations in parameters such as temperature and pH. The release of fragments can also be modulated by chemical interactions, such as the use of an additive (e.g. a second, more polar counterion agent in the stripping solvent capable of competing with the first counterion to form a complex with the RNA molecules, thereby promoting the release of the molecules from the surface of the medium).

The temperature at which the separation is performed affects the choice of organic solvents used in the separation. One reason is that the solvents affect the temperature at which a polynucleotide becomes denatured, losing secondary and tertiary structure, which can affect affinity for the separation medium. Some solvents can stabilize such structure better than other solvents. The other reason a solvent is important is because it affects the distribution of the polynucleotide between the mobile phase and the stationary phase. Acetonitrile and 1-propanol are preferred solvents in these cases. Finally, the toxicity (and cost) of the solvent can be important. In this case, methanol is preferred over acetonitrile and 1-propanol is preferred over methanol.

The process of the invention preferably includes precautions to prevent contamination with multivalent cations such as Fe(III), Cr(III), or colloidal metal contaminants. Multivalent cations can cause non-specific binding of the DNA to the surfaces of conduits and containers which can lead to low recovery. The inner surfaces, which contact liquids within the system, preferably are treated to remove multivalent contaminants, e.g. treating with an acid such as nitric acid. The efficiency of the separation process may be enhanced by the optional addition of a chelating agent such as EDTA, e.g. at a concentration of 0 to 0.1 M. Suitable precautions are described in U.S. Pat. No. 5,772,889. Precautions can also be taken during the manufacture of the separation medium to prevent contamination with multivalent cations. Examples of suitable precautions in the manufacture of beads, for example, are described in U.S. Pat. No. 6,056,877.

Polynucleotides in solution can be detected by any suitable method, e.g., by UV absorbance, fluorescence or radioactivity.

The general process of separating and/or purifying polynucleotides includes the following steps:

1. Loading a solution containing polynucleotides of interest onto separation medium under conditions that promote binding of the polynucleotides of interest. These conditions are typically achieved by including in the solution an appropriate counterion agent and no organic solvent, or a concentration of organic solvent below that which is required to cause elution of the polynucleotides of interest.

2. If desired, non-target polynucleotides can be eluted from the column by means of a wash solution containing a counterion agent and a concentration of organic solvent sufficient to elute the non-target polynucleotides, but insufficient to elute target polynucleotides (optional).

3. Eluting target polynucleotides from the separation medium by application of an elution solution containing sufficient organic solvent to cause the release of the target polynucleotides.

4. The eluted polynucleotides can be collected for further processing or analysis (optional).

5. The eluted polynucleotides can be detected (optional).

6. Steps 3-5 can optionally be repeated one or more additional times, resulting in fractionation of target polynucleotides into multiple fractions.

In practicing the instant invention, the geometry, volume and configuration of the container supporting the separation medium can be varied without loss of the ability to predictably separate polynucleotides on the basis of the physical characteristics, including size and base composition. The container can be, for example, a low-pressure column, a spin column, a web, a pad, a flask, a well, or a tank.

In one embodiment of the invention, separation can be achieved in a batch process. In this embodiment, a relatively polar sample solution containing polynucleotide, including a counterion agent, are mixed in bulk with separation beads in a container, whereby polynucleotides of interest bind to the beads. Preferably, all of the polynucleotide-counterion aggregates will bind nonspecifically to the beads under the initial loading conditions. To release polynucleotides from the beads, the beads are brought into contact with an elution solution with a sufficient concentration of organic solvent to effect elution of the desired polynucleotides. Elution conditions for specific polynucleotides, or classes of polynucleoitdes (e.g., genomic DNA, plasmid DNA, DNA fragments of defined length molecules, mRNA molecules) can often be predetermined, e.g. by determining the elution profile of a standard polynucleotide mixture at various concentrations of organic solvent. This calibration procedure can be conducted on a small scale and applied to a large-scale process. An example of the high resolution which can be obtained in a single equlibria bulk process, in the context of DNA, is exemplified by referring to FIG. 14 and EXAMPLE 9, where isolation of a 102 base pair fragment was achieved by incrementally increasing the ACN concentration from 14.6% to 15.9%.

In a preferred embodiment of the process of the invention, after the sample mixture is bound, a wash solution is applied in a first release step in which the organic solvent is applied at a concentration which will release non-target polynucleotides. The beads are then separated from the solvent, e.g. by centrifugation or by filtration. An elution solution is then applied to the beads in a second release step in which the elution solution contains an incrementally elevated concentration of organic solvent, which selectively releases the target polynucleotide, plurality of polynucleotides, or target class of polynucleotides (e.g., mRNA molecules). Optionally, the process can be repeated with the application of elution solutions containing increasing concentrations of organic solvent in order to successively release polynucleotide fractions characterized by increasing affinity for the separation medium. Each fraction can be recovered, e.g. by collecting the elution solution at each concentration of organic solvent. It is possible to have multiple wash steps at a single concentration of organic solvent to ensure complete removal of target molecules.

In another example of a batch process of the present invention, the separation is performed using a column, e.g. an open column under gravity flow conditions or a low pressure column equipped with a peristaltic pump. The separation medium comprises beads having a diameter large enough to permit flow of stripping solvent without requiring high pressure pumps. Preferred beads have a diameter of about 20 to 1000 microns and can be made from various materials as described hereinabove. The dimensions of the column can range from about 10 cm to 1 m in length, and 1 to 100 cm in diameter, for example. In operation, the column is first conditioned using a polar solvent. In the case where the target polynucleotide is RNA, an RNA-counterion mixture is applied to the column in a convenient volume such as from 1 to 50 mL. For dilute samples having a large volume, the sample can be applied continuously, or in stages, to “load” the column. Preferably, all of the RNA-counterion aggregate will bind to the separation medium under the initial conditions in which the loading solution has low concentration of organic solvent. To release target RNA molecules from the separation beads, the beads are brought into contact with an elution solution having a sufficient concentration of organic solvent. Elution conditions for specific RNA molecules, or classes of RNA molecules, can be pre-determined, e.g. by determining the elution profile of a standard RNA mixture at various concentrations of solvent. This calibration procedure can be conducted on a small scale and applied to a large-scale process. Specific solvent compositions can be adjusted to elute a target RNA in analogy to the bulk equilibria process as described hereinabove. After the sample mixture is bound to the separation medium in the column, a wash solution can be applied in a first release step in which the organic solvent is present at a concentration which will release non-RNA contaminating species (e.g. macromolecules such as proteins, DNA molecules or carbohydrates) and/or non-target RNA molecules having less affinity for the separation medium than target RNA molecules; an elution solution is then applied in a second release step in which the organic solvent is present at an elevated concentration, e.g. an incrementally elevated concentration, which selectively releases the target RNA molecule. Optionally, organic solvents can be applied in a gradient of increasing concentration, e.g. a step-gradient or continuous gradient, in order to progressively release RNA molecules having increasing affinity for the separation medium. Each fraction is recovered, for example, by collecting the elution solution at each concentration of organic solvent. For each fraction, the separation process can be repeated, if necessary, e.g. by application to another column.

In another embodiment of the invention, the separation medium can be retained in a web or pad. An example is a web of inert fiber matrix with hydrophobic separation medium, such as the beads as described hereinabove, enmeshed in the matrix. The web of the present invention is a composite article comprising separation medium which has been incorporated into a fabric or membrane. The term “incorporated into a fabric membrane” means that the separation medium is encapsulated by or trapped within a fabric or membrane, is stabilized within a fabric or membrane or is covalently attached to a fabric or membrane such that the separation medium does not exist as free flowable particulate bulk material and is not separable from the web under liquid chromatography conditions.

In another embodiment of the invention, the separation medium is incorporated into a web, which may be woven or non-woven. The spaces between fibers of the web should be small enough to prevent separation medium material from passing through the web. The density of non-woven fibers and the density of warp and weft fibers of the web can be routinely adjusted to provide the desired density and porosity.

The web fibers can be made of any suitable material so long as the material is porous. Suitable materials are described in U.S. Pat. No. 5,338,448. Generally, the fibers will be made of a porous synthetic or natural polymeric material, e.g. polytetrafluoroethylene, cellulose, polyvinyl chloride, nylon, etc. The RNA in the sample preferably binds only to the separation medium and the binding is not detrimentally affected by the fiber matrix material. When the separation medium consists of polymeric beads, the ratio of beads to fiber matrix material can be in the range of 19:1 to 4:1 by weight, for example.

In one embodiment, the web is mounted on a support and the sample is applied and eluted in a manner analogous to the open column process as described hereinabove. The web material can be packed into a column. An advantage of using a web material is that it provides flexibility in how thin a column bed can be made, e.g. the web can be formed as a disk. Also, several uniform beds can be made at once. Multiple webs can be supported in a row or adapted to a matrix well format, e.g. a multi-well plate. The web can be used in analogy to the bulk equilibria process or column as described hereinabove with a binding step followed by release steps.

An example of a suitable fibril matrix is polytetrafluoroethylene (PTFE) as described in U.S. Pat. No. 4906378 to Hagen. The ratio of beads to PTFE fibril matrix can be in the range of 19:1 to 4:1 by weight, for example.

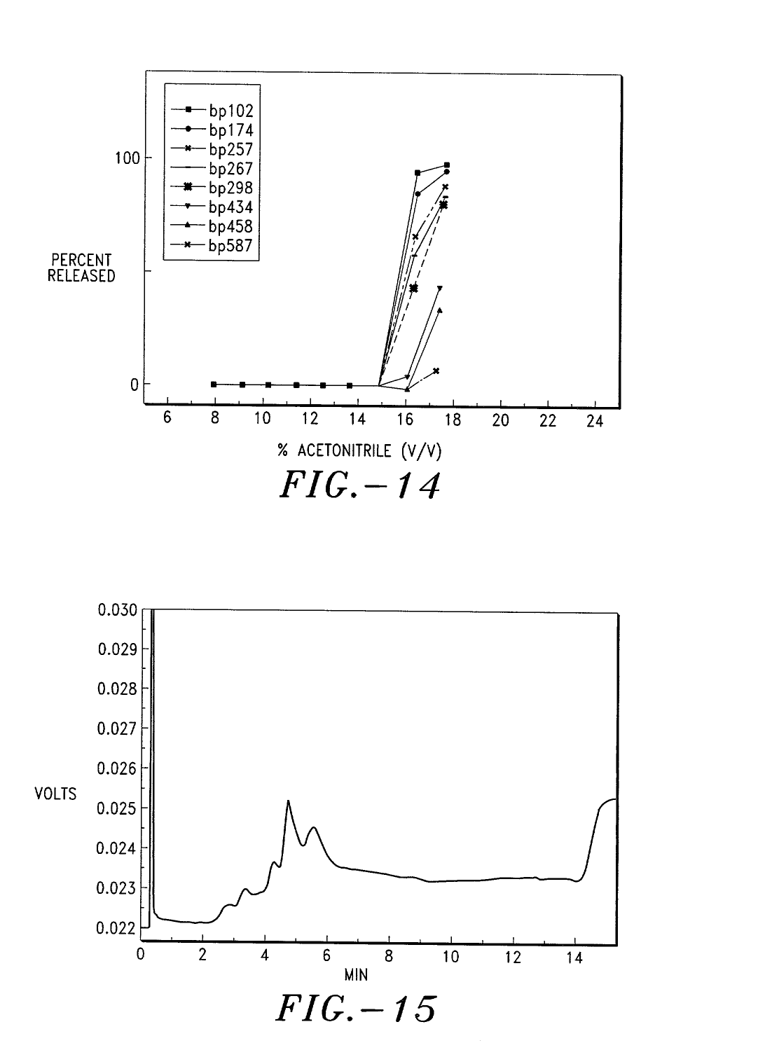

Referring to FIG. 15 and EXAMPLE 10, a DNA fragment separation was performed using discs of 0.7 mm thickness, and demonstrated that separation is possible using a thin separation bed containing hydrophobic separation medium.

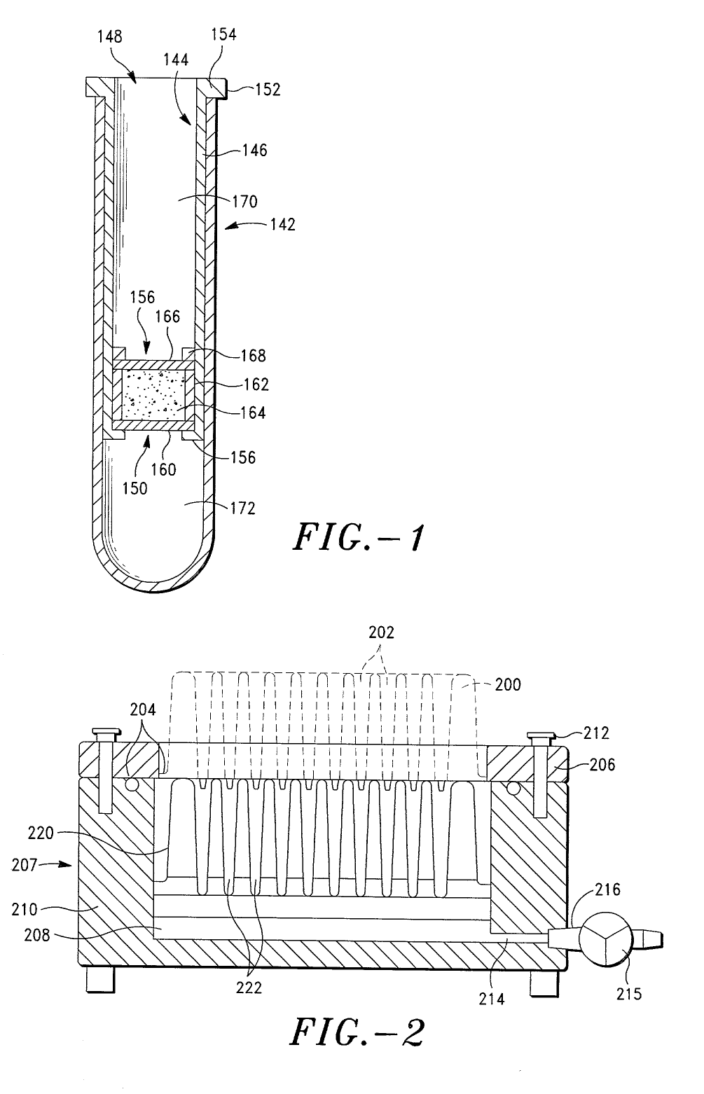

In another embodiment of the invention, separation is achieved by means of a spin column. FIG. 1 is a cross-sectional view of a spin column separation device suitable for such a use. In this embodiment a standard laboratory centrifuge is used to rapidly pass liquids through the separation medium. The system uses a standard cylindrical centrifuge vial or eluant container 142 into which a separator tube or cylinder 144 is inserted. The separator cylinder can have a cylindrical body 146, open at top end 148 and bottom end 150, and sized to fit within the vial 142. The upper end 148 has an outwardly extending upper flange 152 which is sized to rest on the upper rim 154 of the cylindrical vial 142. The lower end 150 has an inwardly extending lower flange 156 which is sized to support the separation unit 158.

The separation unit comprises a porous support disk 160 which rests on flange 156, an optional outer cylinder 162 within which the separation medium 164 is positioned. The separation unit can also comprise an optional upper porous disk 166 to prevent disruption of the separation medium and an optional ring 168. The optional ring 168 preferably has a slightly elastic or yielding composition and an outer diameter which is sized to establish a frictional engagement with the inner wall of cylinder 146. The ring 168, when pressed against the disk 166, holds the disk in place during use of the column.

The separation of DNA and RNA molecules using the device of FIG. 1 are demonstrated in the Examples presented hereinbelow. In general, polynucleotide separations can be achieved by applying the following sequence of non-limiting steps.

1. A solution containing the polynucleotide (or polynucleotides) of interest is diluted in a loading solution containing an appropriate counterion agent and no organic solvent, or a concentration of organic solvent below that which is required to cause elution of the polynucleotide of interest.

2. The diluted mixture is placed into chamber 170 and the spin column is placed in a standard laboratory centrifuge and spun until all of the free liquid has passed into the chamber 172. The inner cylinder 144 is removed from the vial, and the contents of chamber 172 are discarded (or saved if so desired). The polynucleotides to be separated bind to the separation medium in this step.

3. If so desired, a wash solution containing counterion and an organic solvent is added to chamber 170. The organic solvent concentration is calculated to be the amount which will remove non-target molecules that have less affinity for the separation medium than the target polynucleotide. The appropriate concentration of organic solvent can be pre-determined as described supra.

4. If a wash step is used, the separation device is spun in a centrifuge until all of the free wash solution has passed into the chamber 172. The inner cylinder 144 is removed from the vial, and the contents of chamber 172 are removed. This step removes from the separation medium contaminants and other non-target molecules that have less affinity for the separation medium than the target polynucleotide.

5. An elution solution containing counterion and a higher concentration of organic solvent is prepared and placed in the chamber 170. The concentration of organic solvent is calculated to be the amount which will remove target polynucleotides from the separation medium. In some instances, it will be desirable to use a concentration of organic solvent that is low enough to cause non-target molecules with greater affinity for separation medium than the target polynucleotide to remain bound to the column, thereby effectively separating these molecules from target polynucleotide.

6. The separation device is centrifuged until all of the free elution solution has passed into the chamber 172. The inner cylinder 144 is removed from the vial, and the contents of chamber 172, containing purified target RNA molecule or molecules, is removed for further processing.

Obviously, vial 142 can be replaced between steps or cleaned between steps to prevent contamination of the product fraction or fractions.

The concentration of organic solvent in the elution solution can be selected to remove a single polynucleotide species or a plurality of polynucleotides sharing similar physical characteristics and hence affinity for the separation medium.

Steps (5) and (6) can be repeated with successively higher concentrations of organic solvent to remove a series of polynucleotide-containing fractions.

It will be readily apparent to a person skilled in the art that other variations can be applied to remove a one or a series of purified fractions in much the same manner as is shown above and illustrated in the Examples and Figures of this application.

In another embodiment of the invention, separation is achieved by means of a vacuum tray separation device. FIG. 2 is a cross-sectional view of a vacuum tray separation device suitable for use in this invention, and FIG. 3 is a top view of the separation tray of FIG. 3. The separator tray 200 is a single plate with rows and columns of tubular separation channels 202, preferably having regular, repeated spaces between the rows and columns for indexing the spaces. The dimensions of the tray 200 and separation channels can correspond and match the dimensions of standard multi-well plates, such as the 96 cavity microtiter plate.

The multicavity separation plate 200 is supported on support flange and vacuum seals 204 formed in the internal cavity of an upper plate 206 of the vacuum assembly 207. The vacuum assembly 207 further comprises a vacuum cavity 208 defined by housing 210. The upper plate 206 positioned on the housing 210 by locating pins 212, and the upper plate 206 and the housing 210 have a sealed engagement with the seals 204. The housing 210 has an exhaust outlet channel 214 communicating with the vacuum chamber 208 and with a vacuum conduit 216 and vacuum valve 218. The vacuum conduit 216 and vacuum valve 218 communicate with a vacuum source (not shown).

A multi-well collection plate 220 is supported in the vacuum chamber 208. The multi-well collection plate 220 is a single plate with rows and columns of separation channels 222, preferably having regular, repeated spaces between the rows and columns for indexing the spaces. The dimensions of the tray 220 and collection channels can correspond and match the dimensions of standard multi-well plates, such as the 96 cavity microtiter plate. The collection plate 220 is held in a position which aligns each of the collection wells 222 with a corresponding separating channel 202 of the separation plate 200 so each well 222 can collect liquid falling from the corresponding separation channel 202.

FIG. 4 is a cross-sectional view of the separation tray of FIG. 3 taken along the line A-A. The separation channels 202 each have an evenly spaced upper cavity 224, separation medium 226 and a liquid outlet 228.

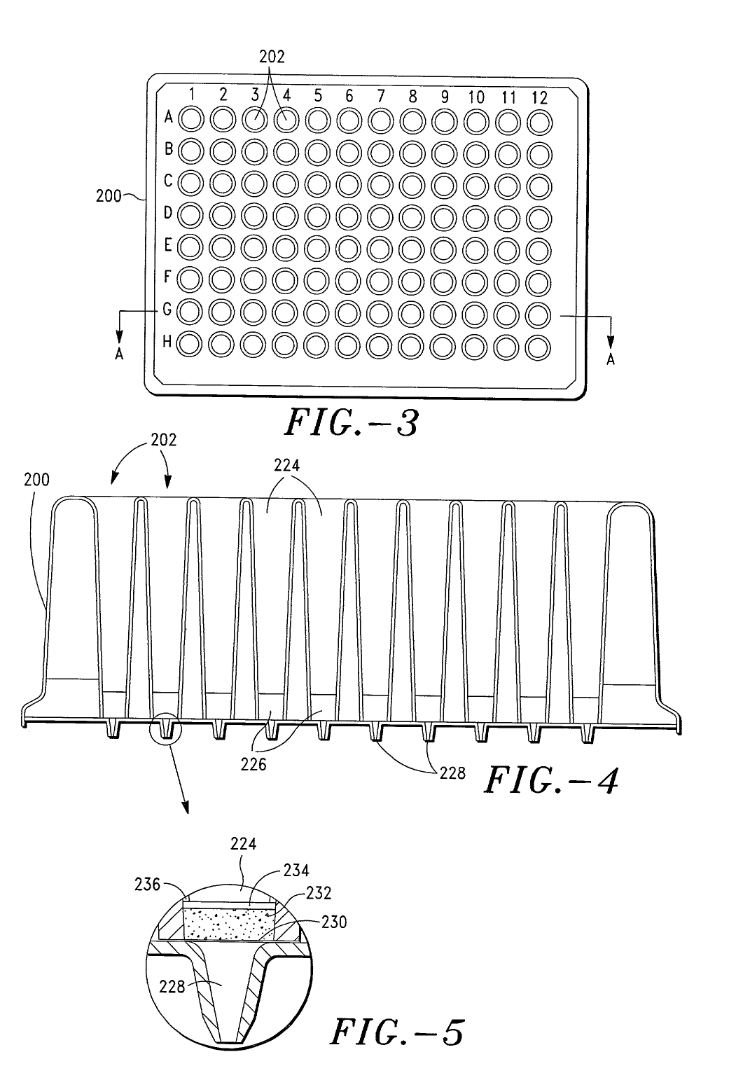

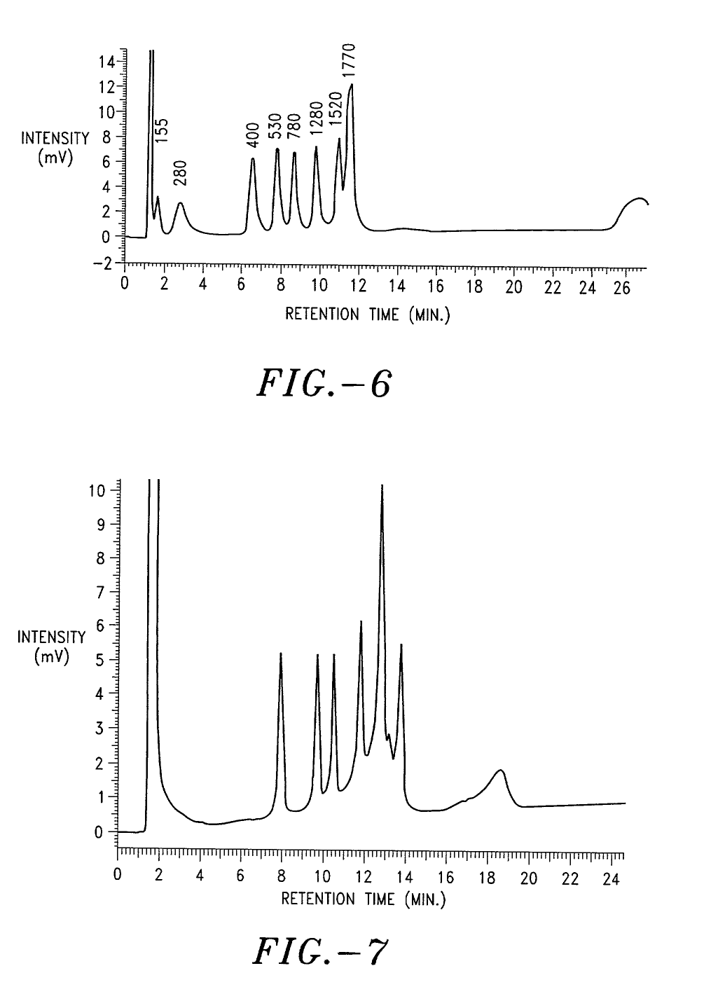

FIG. 5 is an enlarged view of the separation components of the separation tray of FIG. 4. The bottom of the separation cavity 224 supports a porous disk 230, which in turn supports separation medium 232. An optional containment disk 234 rests on the separation medium 232, and the containment disk 234 can be optionally held in place by friction ring 236 or an equivalent device.

The separation medium 232 can be the same nonpolar medium as described above.

The separation of polynucleotides using the device of FIGS. 2-5 can be achieved by the following sequence of steps.

1) A loading solution is prepared containing the polynucleotide (or polynucleotides) of interest, an appropriate counterion agent and no organic solvent, or a concentration of organic solvent below that which is required to cause elution of the polynucleotide of interest.

2) The loading solution is placed in one of the chambers 202 of the fully assembled vacuum device. The other chambers 202 are filled with other polynucleotide containing loading solutions to be separated by the same procedure.

3) Vacuum is applied to the vacuum chamber 208 by opening vacuum valve 218 until all of the liquid from the mixtures contained in each chamber has collected in chambers 222. The vacuum device is disassembled, and the contents of chambers 222 are discarded. The polynucleotides to be separated bind to the separation medium 232 in each chamber 202 in this step.

4) The vacuum apparatus and plates are reassembled, and a wash solution containing counterion and an organic solvent is added to the chambers 202. The organic solvent concentration is calculated to be the amount which will remove non-target molecules that have less affinity for the separation medium than the target polynucleotide. The appropriate concentration of organic solvent can be pre-determined as described supra.

5) Vacuum is applied to the vacuum chamber 208 by opening vacuum valve 218 until all of the liquid from the mixtures contained in each chamber has collected in chambers 222. The vacuum device is disassembled, and the contents of chambers 222 are removed This step removes from the separation medium contaminants and other non-target molecules that have less affinity for the separation medium than the target polynucleotide.

6) The vacuum apparatus and plates are reassembled, and an elution solution containing counterion and an organic solvent is placed in the chambers 202. The concentration of organic solvent is calculated to be the amount which will remove target polynucleotides from the separation medium. In some instances, it will be desirable to use a concentration of organic solvent that is low enough to cause non-target molecules with greater affinity for separation medium than the target polynucleotide to remain bound to the column, thereby effectively separating these molecules from target polynucleotide.

7) Vacuum is applied to the vacuum chamber 208 by opening vacuum valve 218 until the liquid from the mixtures contained in each chamber has collected in chambers 222. The vacuum device is disassembled, and the contents of chambers 222, containing purified target polynucleotide or polynucleotides of interest, is removed for further processing.

Obviously, the plate 220 can be replaced between steps or cleaned between steps to prevent contamination of the product fraction or fractions.

The concentration of organic solvent in the elution solution can be selected to remove a single polynucleotide or a genus of polynucleotides sharing similar physical characteristics and hence affinity for the separation medium.

Steps (6) and (7) can be repeated with successively higher concentrations of organic solvent to remove a series of polynucleotide fractions.

It will be readily apparent to a person skilled in the art that other variations can be applied to remove a series of purified fractions in much the same manner as is shown above and illustrated in the Examples and Figures of this application.

The spin column components 142 and 146 of FIG. 1 and the plates 200 and 220 in FIGS. 2-5 are made of a material which does not interfere with the separation process such as polystyrene, polypropylene, or polycarbonate. The upper plate 206 and housing 210 can be made of any materials having the requisite strength such as a rigid organic polymer, aluminum, stainless steel or the like. The vacuum chamber walls are preferably coated with Teflon film. The vacuum conduit and valve can also be made of Teflon coated aluminum or the like.

This invention is further illustrated by the following specific but non-limiting examples where the descriptions of methods in the past tense represent completed laboratory experiments. Descriptions in the present tense have not been carried out in the laboratory and are herein constructively reduced to practice by the filing of this application. All references referred to herein, including any patent, patent application or non-patent publication, are hereby incorporated by reference in their entirety.

### EXAMPLE 1

### Preparation of Nonporous Poly(styrene-divinylbenzene) Particles

Sodium chloride (0.236 g) was added to 354 mL of deionized water in a reactor having a volume of 1.0 liter. The reactor was equipped with a mechanical stirrer, reflux condenser, and a gas introduction tube. The dissolution of the sodium chloride was carried out under inert atmosphere (argon), assisted by stirring (350 rpm), and at an elevated temperature (87° C.). Freshly distilled styrene (33.7 g) and 0.2184 g of potassium peroxodisulfate (K₂S₂O₈) dissolved in 50 mL of deionized water were then added. Immediately after these additions, the gas introduction tube was pulled out of the solution and positioned above the liquid surface. The reaction mixture was subsequently stirred for 6.5 hours at 87° C. After this, the contents of the reactor were cooled down to ambient temperature and diluted to a volume yielding a concentration of 54.6 g of polymerized styrene in 1000 mL volume of suspension resulting from the first step. The amount of polymerized styrene in 1000 mL was calculated to include the quantity of the polymer still sticking to the mechanical stirrer (approximately 5-10 g). The diameter of the spherical beads in the suspension was determined by light microscopy to be about 1.0 micron.

Beads resulting from the first step are still generally too small and too soft (low pressure stability) for use as chromatographic packings. The softness of these beads is caused by an insufficient degree of crosslinking. In a second step, the beads are enlarged and the degree of crosslinking is increased.

The protocol for the second step is based on the activated swelling method described by Ugelstad et al. (Adv. Colloid Interface Sci., 13:101-140 (1980)). In order to initiate activated swelling, or the second synthetic step, the aqueous suspension of polystyrene seeds (200 ml) from the first step was mixed first with 60 mL of acetone and then with 60 mL of a 1-chlorododecane emulsion. To prepare the emulsion, 0.206 g of sodium dodecylsulfate, 49.5 mL of deionized water, and 10.5 mL of 1-chlorododecane were brought together and the resulting mixture was kept at 0° C. for 4 hours and mixed by sonication during the entire time period until a fine emulsion of<0.3 microns was obtained. The mixture of polystyrene seeds, acetone, and 1-chlorododecane emulsion was stirred for about 12 hours at room temperature, during which time the swelling of the beads occurred. Subsequently, the acetone was removed by a 30 minute distillation at 80° C.

Following the removal of acetone, the swollen beads were further grown by the addition of 310 g of a ethyldivinylbenzene and divinylbenzene (DVB) (1:1.71) mixture also containing 2.5 g of dibenzoylperoxide as an initiator. The growing occurred with stirring and with occasional particle size measurements by means of light microscopy.

After completion of the swelling and growing stages, the reaction mixture was transferred into a separation funnel. In an unstirred solution, the excess amount of the monomer separated from the layer containing the suspension of the polymeric beads and could thus be easily removed. The remaining suspension of beads was returned to the reactor and subjected to a stepwise increase in temperature (63° C. for about 7 hours, 73° C. for about 2 hours, and 83° C. for about 12 hours), leading to further increases in the degree of polymerization (>500). The pore size of beads prepared in this manner was below the detection limit of mercury porosimetry (<30 Å).

After drying, the dried beads (10 g) from step two were washed four times with 100 mL of n-heptane, and then two times with each of the following: 100 mL of diethylether, 100 mL of dioxane, and 100 mL of methanol. Finally, the beads were dried.

### EXAMPLE 2

### Alkylation of Poly(Styrene-Divinylbenzene) Polymer Beads

The following procedures were carried out under nitrogen (Air Products, Ultra Pure grade, Allentown, Pa.) at a flow rate of 250-300 mL/min. 25 g of the beads prepared in Example 1 were suspended in 150-160 g of 1-chlorooctadecane (product no. 0235, TCI America, Portland, Ore.) using a bow shaped mixer (use a 250 mL wide neck Erlenmeyer flask). The temperature was set to 50-60° C. to prevent the 1-chlorooctadecane from solidifying. Larger pieces of polymer were broken up to facilitate suspending. The solution was mixed using a stirrer (Model RZRI, Caframo, ONT NOH2T0, Canada) with the speed set at 2. The polymer suspension was transferred into a three neck bottle (with reflux condenser, overhead stirrer and gas inlet). 52-62 g of 1-chlorooctadecane were used to rinse the Erlenmeyer flask and were added to the three neck bottle. The bottle was heated in an ethylene glycol bath set at 80° C. The solution was mixed using a stirrer (Caframo) with the speed set at 0. After 20 minutes, the reaction was started by addition of 1.1 g AlCl₃ powder (product no. 06218, Fluka, Milwaukee, Wis.) and continued for 16-18 h.

After the reaction, the polymer was separated from excess 1-chlorooctadecane by centrifugation followed by consecutive washing steps:

| Addition | Comment |
|---|---|
| 50 mL conc. HCl, 50-60 mL | 4 repetitions, with recycled heptane |
| n-heptane | |
| 100 mL H₂O, 50-60 mL n-heptane | 1 repetition, with fresh heptane |
| 50 mL conc. HCl, 50-60 mL | 1 repetition, with fresh heptane |
| n-heptane | |
| 100 mL H₂O, 50-60 mL n-heptane | 1 repetition, fresh heptane |
| 150 mL H₂O, no n-heptane | 3 repetitions, use plastic stirrer to |
| | break up chuncks of polymer |
| | beads. Repeat steps 4 and 5 three |
| | times. Shake for two minutes with |
| | no centrifugation. |
| 100 mL THF | 3 repetitions |
| 100 mL THF/n-heptane | 1 repetition |
| 100 mL n-heptane | 1 repetition |
| 100 mL THF | 1 repetition |
| 100 mL CH₃OH | 4 repetitions |

In the steps where aqueous solvents (HCl or H₂O) were used, the polymer was shaken for 30 seconds with the aqueous phase before adding n-heptane. n-Heptane was then added and the mixture was shaken vigorously for 2 min. After the final polymeric beads were dried at 40-50° C. for 2-3 hr, they were ready for packing.

### EXAMPLE 3

### Acid Wash Treatment

The beads prepared in Example 2 were washed three times with tetrahydrofuran and two times with methanol. Finally the beads were stirred in a mixture containing 100 mL tetrahydrofuran and 100 mL concentrated hydrochloric acid for 12 hours. After this acid treatment, the polymer beads were washed with a tetrahydrofuran/water mixture until neutral (pH=7). The beads were then dried at 40° C. for 12 hours.

### EXAMPLE 4

### RNA Segregation of an RNA Sizing Standard by IP-RP-HPLC using a 7.8 mm ID Column

IP-RP-HPLC analysis of a 0.16-1.77 Kb RNA ladder (Catalog no. 15623010, Life Technologies) was performed using C-18 alkylated nonporous poly(styrene-divinylbenzene) beads packed in a 50 mm×7.8 mm ID column (DNASEP® cartridge, Transgenomic, Inc., San Jose, Calif.) and using a WAVE® Nucleic Acid Fragment Analysis System (Transgenomic). Buffer A: 0.1 M TEAA, pH 7.0; buffer B: 0.1 TEAA, 25% (v/v) acetonitrile, pH 7.0. The buffer stock solutions were obtained form Transgenomic. The gradient conditions were as follows:

| Time (min) | % B |
|---|---|
| 0.0 | 38 |
| 1.0 | 40 |
| 16 | 60 |
| 22 | 66 |
| 22.5 | 70 |
| 23 | 100 |
| 24 | 100 |
| 25 | 38 |
| 27 | 38 |

The flow rate was 0.9 mL/min and the column temperature was 75.0° C. UV detection was performed at 260 nm. The injection volume was 5.0 μL. The sample contained a mixture of eight RNAs having the nucleotide lengths as shown in FIG. 6.

Prior to the injection, the column was equilibrated with 75% acetonitrile for 30-45 min at a flow rate of 0.9 mL/min. The column was then equilibrated using 38% B for 30 min. Prior to the elution of RNA, two control gradient elutions (using the same gradient conditions as for the RNA) were performed: a first injection of 10 μL of 0.5 mM EDTA and a second injection of 10 μL of nuclease free water (Catalog no. 9930, Ambion, Inc., Austin, Tex.). These two injections (data not shown) demonstrated that the column was free from contamination.

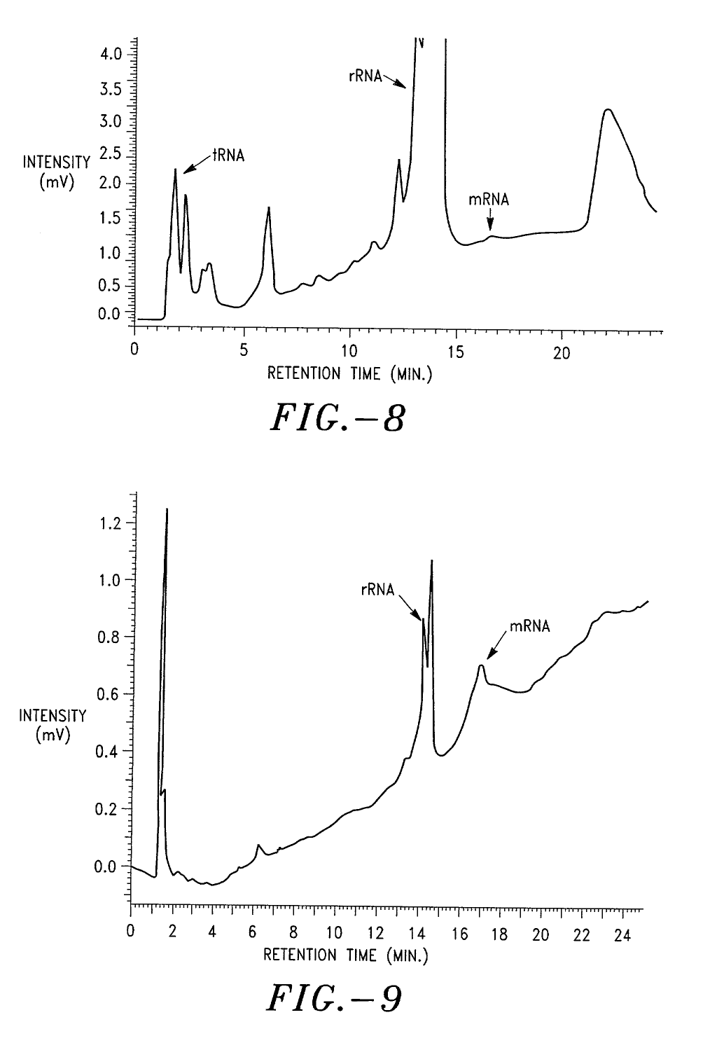

Another sizing standard (catalog no. 1062611, Roche Molecular Biochemicals, Indianapolis, Ind.) was similarly analyzed as shown in FIG. 7. 1 μg RNA was injected in a volume of 1 μL.

In preparing the mRNA sample for injection, all chemicals were of the highest purity grade available for molecular biology. Solutions, glassware, and small instruments were sterilized whenever possible. Liquid transfers were made using RNase free pipette tips (Rainin Instrument Co., Inc., Woburn, Mass.). All manipulations were performed wearing surgical gloves.

### EXAMPLE 5

### RNA Segregation of Tobacco Plant RNA by IP-RP-HPLC using a 7.8 mm ID Column

Total RNA was extracted from the flower of tobacco plant (Nicotiana tabacum cv. Wisconsin 38) by an acid guanidinium thiocyanate phenol-chloroform extraction method, and precipitated with 4 M lithium chloride (Chomczynski, et al. (1987) Anal. Biochem. 162:156-159) as described in Bahrami, et al. (1999) Plant Molecular Biology 39:325-333.

IP-RP-HPLC analysis of total RNA from the plant extract was performed using C-18 alkylated nonporous poly(styrene-divinylbenzene) beads packed in a 50 mm×7.8 mm ID column (DNASEP® cartridge, Transgenomic, Inc., San Jose, Calif.) and using a WAVE® Nucleic Acid Fragment Analysis System (Transgenomic). Buffer A: 0.1 M TEAA, pH 7.0; buffer B: 0.1 TEAA, 25% (v/v) acetonitrile, pH 7.0. The gradient conditions were as described in Example 4. The volume injected was 2 μL (containing 1.54 μg RNA). The chromatogram is shown in FIG. 8.

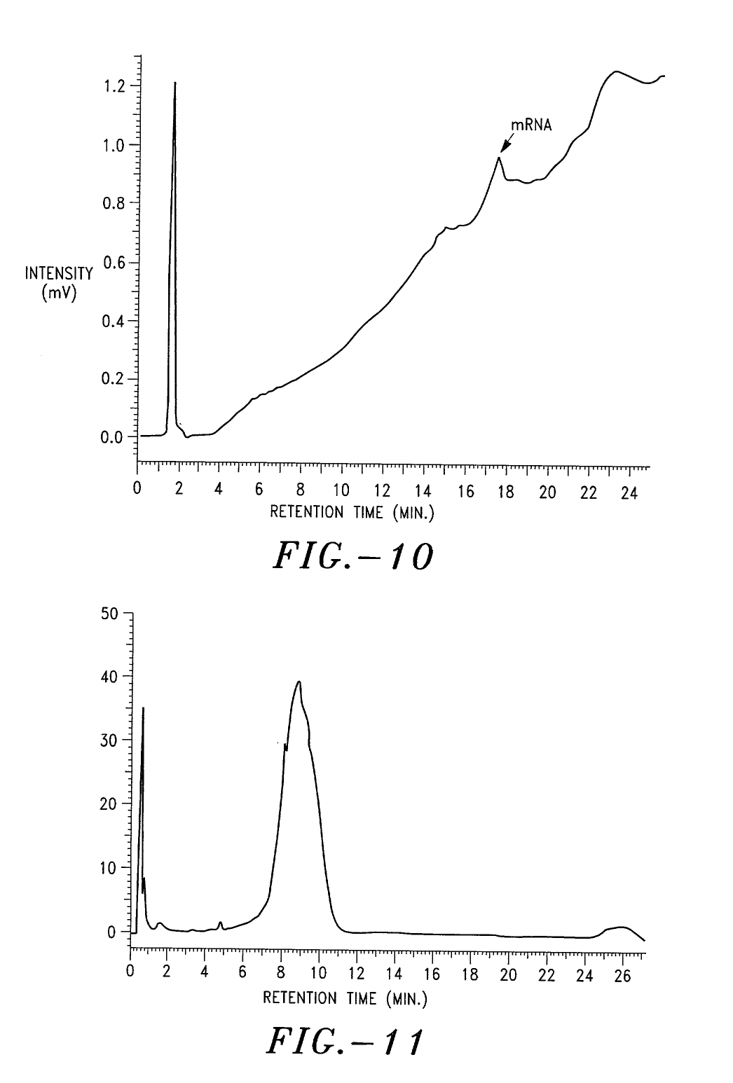

mRNA was extracted from 50 μg of the total RNA preparation using the OLIGOTEX mRNA Purification System from Qiagen and following the procedures supplied with the kit (catalog no. 70022). A portion of the extracted mRNA was analyzed by IP-RP-HPLC (FIG. 9) using the elution conditions described in Example 4. The product of the first OLIGOTEX extraction was re-extracted, and a portion of the product was again analyzed by IP-RP-HPLC (FIG. 10).

### EXAMPLE 6

### RNA Segregation of Mouse Brain mRNA by IP-RP-HPLC using a 7.8 mm ID Column

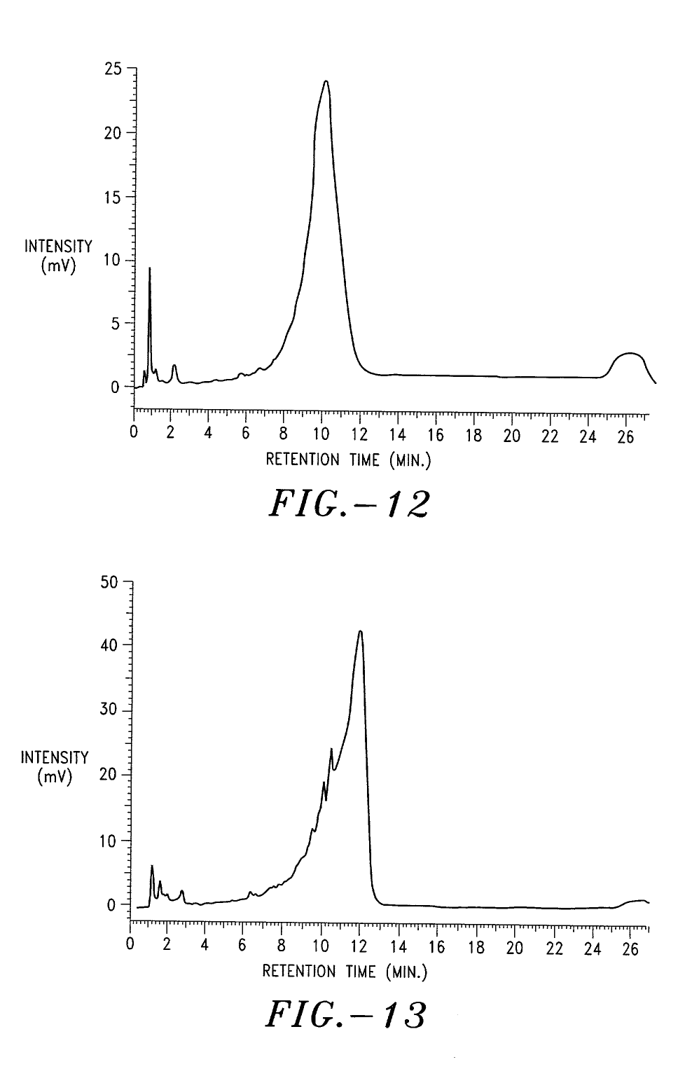

5 μL (4.5 μg RNA) of mouse brain mRNA was subjected to IP-RP-HPLC analysis, using elution conditions as described in Example 4, with the resulting chromatograph shown in FIG. 11.

### EXAMPLE 7

### RNA Segregation of Human Brain mRNA by IP-RP-HPLC using a 4.6 mm ID Column

IP-RP-HPLC analysis of human brain mRNA (Catalog no. 6516-1, Clontech Laboratories, Inc., Palo Alto, Calif.) was performed using C-18 alkylated nonporous poly(styrene-divinylbenzene) beads packed in a 50 mm×4.6 mm ID column (DNASEP® cartridge, Transgenomic, Inc., San Jose, Calif.) and using a WAVE® Nucleic Acid Fragment Analysis System (Transgenomic). Buffer A: 0.1 M TEAA, pH 7.0; buffer B: 0.1 TEAA, 25% (v/v) acetonitrile, pH 7.0. The gradient conditions were as follows:

| Time (min) | % B |
|---|---|
| 0.0 | 38 |
| 1.0 | 40 |
| 16 | 60 |
| 22 | 66 |
| 22.5 | 70 |

The flow rate was 0.9 mL/min and the column temperature was 75.0° C. UV detection was performed at 260 nm. Injection volume was 4.5 μL. The chromatogram is shown in FIG. 12.

### EXAMPLE 8

### RNA Segregation of Human Brain mRNA by IP-RP-HPLC using a 7.8 mm ID Column

IP-RP-HPLC analysis was performed using the same mRNA sample and conditions as described in Example 7 except that the column was replaced by a 50 mm×7.8 mm ID column. The injection volume was 5.5 μL. The chromatogram is shown in FIG. 13.

### EXAMPLE 9

### Separation of DNA Fragments Using a Single Equilibrium Bulk Process

The separation of dsDNA fragments from a pUC18-DNA HaeIII digest was performed using 2.1 micron C-18 alkylated nonporous poly(styrene-divinylbenzene) beads. Nine different vials each containing 0.035 g of beads and 10 μL of DNA digest (4.5 μg) were mixed with 100 μL of 0.1 M triethylammonium acetate (TEAA), each vial containing different amounts of ACN. The incubation time was 10 min at 23° C. The vials were centrifuged with a Brinkman model 3200 table-top centrifuge for 5 minutes. A 3 μL aliquot of the supernatant was removed by syringe for analysis. The analysis was done using HPLC with on-line UV detection at 260 nm. TABLE 1 shows the concentration of DNA, TEAM, ACN and the amount of resin in the different experiments.

TABLE I

| | Amount of resin | Volume | | | DNA |
|---|---|---|---|---|---|
| Exp. | (g) | (μL) | % ACN | TEAA | (μg/μL) |
| 1 | 0.035 | 110 | 7.88% | 0.1 M | 0.0405 |
| 2 | 0.035 | 110 | 9.01% | 0.1 M | 0.0405 |
| 3 | 0.035 | 110 | 10.14% | 0.1 M | 0.0405 |
| 4 | 0.035 | 110 | 11.26% | 0.1 M | 0.0405 |
| 5 | 0.035 | 110 | 12.39% | 0.1 M | 0.0405 |
| 6 | 0.035 | 110 | 13.51% | 0.1 M | 0.0405 |
| 7 | 0.035 | 110 | 14.64% | 0.1 M | 0.0405 |
| 8 | 0.035 | 110 | 15.91% | 0.1 M | 0.0405 |
| 9 | 0.035 | 110 | 17.05% | 0.1 M | 0.0405 |

Referring to FIG. 14, the experiments showed that the smaller fragments (80, 102, 174 bp) in this particular digest were released quantitatively from the resin surface by increasing the ACN concentration from 15 to 16% (in solution) and the larger fragments (257, 267, 298, 434, and 587 bp) by increasing the ACN concentration from 16 to 18.5%. Quantitative release for the 102 bp fragment was achieved by increasing the ACN concentration from 14.6% to 15.9%.

### EXAMPLE 10

### Separation of DNA Fragments Using Discs

FIG. 15 shows the separation of dsDNA fragments from a pUC18-DNA HaeIII digest performed using 8 micron C-18 alkylated nonporous poly(styrene-divinylbenzene) polymer beads in two discs placed in series. The discs are available commercially under the trademark Guard Disc™ (Transgenomic, Inc., San Jose, Calif.) which contain beads enmeshed in a web of TEFLON™ fibril matrix at a weight ratio of 9:1 beads to fibril matrix.

The DNA separation was run under the following conditions: Guard Disc™ 0.7×4.6 mm i.d.; stripping solvent 0.1 M TEAA, pH 7.2; gradient: 35-55% acetonitrile (ACN) in 3 min, 55-65% ACN in 7 min, 65% ACN for 2.5 min; 100% ACN for 1.5 min, back to 35% ACN in 2 min. The flow rate was 0.75 mL/min, detection UV at 260 nm, column temp. 51° C., p=50 psi. The sample was 3 μL (=0.12 μg pUC18 DNA-HaeIII digest).

### EXAMPLE 11

### Separation of PCR Reaction Products Using Discs

The reaction products of a PCR preparation are separated under the conditions as described in EXAMPLE 10. Primer dimers elute in about 2-3 minutes and are well resolved from a 405 base pair PCR product which elutes in about 4-5 minutes.

### EXAMPLE 12

### Spin Column Preparation

A 50 mg portion of resin is added to each spin column (See schematic representation of column in FIG. 1) while pulling a vacuum on the columns. The sides of the columns were tapped to remove resin from the walls. A polyethylene filter was placed on the top of the resin in each vial, followed by a retaining ring, with gentle tapping with a hammer to position the retaining ring securely against the filter. The spin columns were washed with an aqueous solution containing 50% acetonitrile (ACN) and 0.1 M triethylammonium acetate (TEAA). The vials were then washed with an aqueous solution of 25% ACN and 0.1 M TEAA and then with an aqueous solution of 0.1 M TEAA.

### EXAMPLE 13

### Purification of RNA Using a Spin Column

In order to remove free multivalent columns and RNase activity from the spin columns prior to use, the following cleaning protocol was used.

(a) Loaded 600 μL Binding buffer (6.0% ACN, 0.12 M TEAA) onto column

(b) Centrifuged @ 10000 rpm for 1 min.

(c) Loaded 600 μL 0.5M EDTA, pH=8.0 (Research Genetics) onto column.

(d) Centrifuged @ 10000 rpm for 1 min.

(e) Loaded 600 μL DEPC-treated water onto column.

(f) Centrifuged @ 10000 rpm for 1 min.

(g) Loaded 600 μL Binding buffer onto column.

(h) Centrifuged @ 10000 rpm for 1 min.

(i) Loaded 600 μL Binding buffer onto column.

(j) Centrifuged @ 10000 rpm for 1 min.

In order to assess the stability of RNA on the cleaned column, the following steps were performed:

(k) 10 μL mouse brain total RNA sample (10 μg) was mixed with 50 μL Binding buffer.

(l) Centrifuged @ 7000 rpm for 1 min.

(m) Loaded 600 μL Wash buffer (10.0% ACN/0.1M TEAA) onto column.

(n) Centrifuged @ 7000 rpm for 1 min.

(o) Loaded 600 μL Elution buffer (15.0% ACN/0.1M TEAA) onto column.

(p) Centrifuged @ 7000 rpm for 1 min.

No RNA was released from the column during the binding and wash steps, but total RNA was found to be released by Elution buffer.

### EXAMPLE 14

### Separation of PUC 18 MspI with Spin Column

A sample solution was prepared by diluting 35 ml stock pUC 18 Msp I to 1 ml with 0.1 M TEAA (1 ml total volume). A 400 ml aliquot (corresponding to 6.6 mg loaded on the column) was selected. Base pair length separation of the solution was performed using the WAVE separation system (Transgenomic, Inc., Omaha, Neb.) described in FIGS. 1-3, and the chromatograms obtained for two of the columns are shown in FIG. 16 for the pUC 18 Msp I standard.

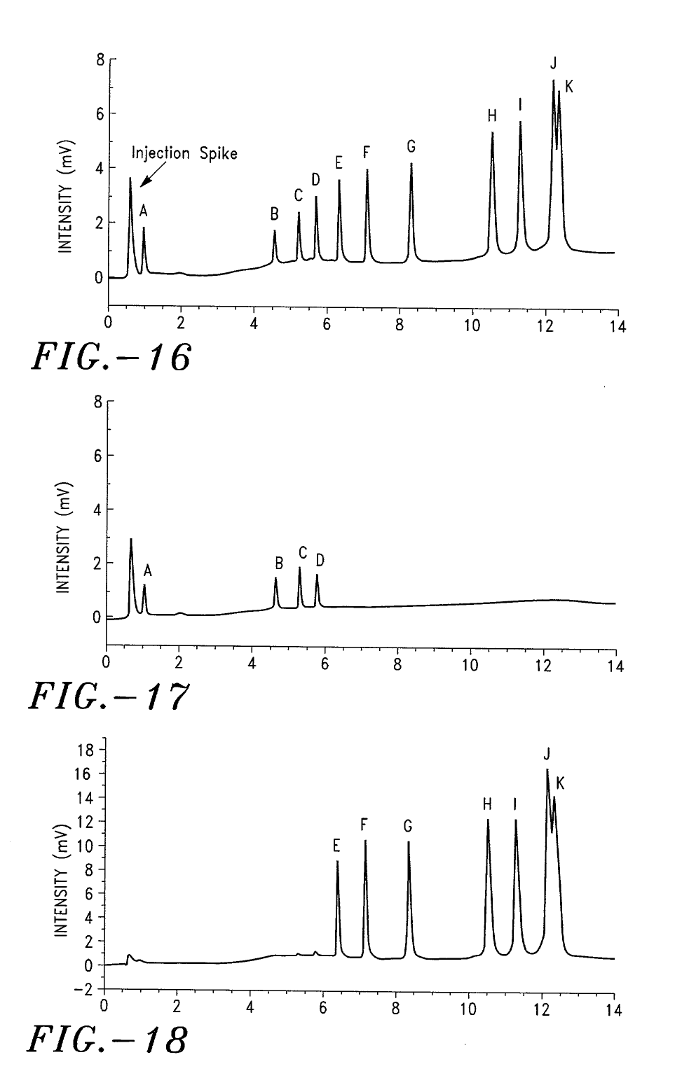

An aliquot of the sample solution was pipetted into separate spin columns and left standing for 5 min. Each vial was centrifuged at 5000 rpm for 5 min. Then 400 μl of freshly prepared aqueous solution containing 9.5% ACN and 0.1 M TEAA was pipetted into each spin column, each vial was left standing for 5 min, each vial was centrifuged at 5000 rpm for 5 min, and the filtrate was analyzed using the WAVE separation system. The chromatogram obtained for the eluant is shown in FIG. 17 for the pUC 18 Msp I standard. The treatment procedure was repeated.

The above procedure was repeated, replacing the 38% B solution with 100 μl of a 100% B solution. The chromatogram by analyzing the eluant with the WAVE separation system is shown in FIG. 18 for the pUC 18 Msp I standard.

This example demonstrates the removal of smaller size fragments from the column while retaining the larger-sized fragments on the column, and subsequent removal of the larger-sized fragments from the column. This is particularly useful for purifying a larger-sized fragment or fragments from smaller size contaminants.

### EXAMPLE 15

### Separation of pBR322HAE III With Spin Column

A sample solution was prepared by diluting 18 ml stock pbr322 HAE III digest to 1 ml with 0.1 M TEAA (1 ml total volume). A 400 ml aliquot (corresponding to 6.6 mg loaded on column) was selected. Base pair length separation of the solution was achieved by IP-RP-HPLC using the WAVE nucleic acid analysis system (Transgenomic, Inc., San Jose, Calif.), and the chromatograms obtained for two of the columns are shown in FIG. 19 for the pBR322HAE III standard.

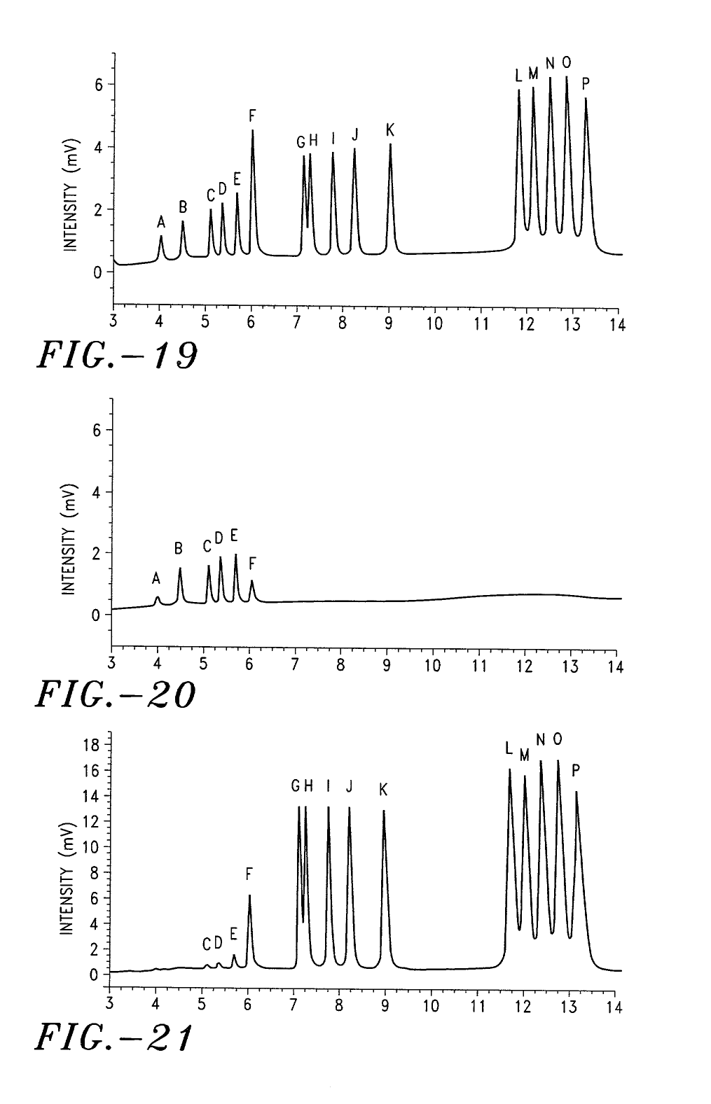

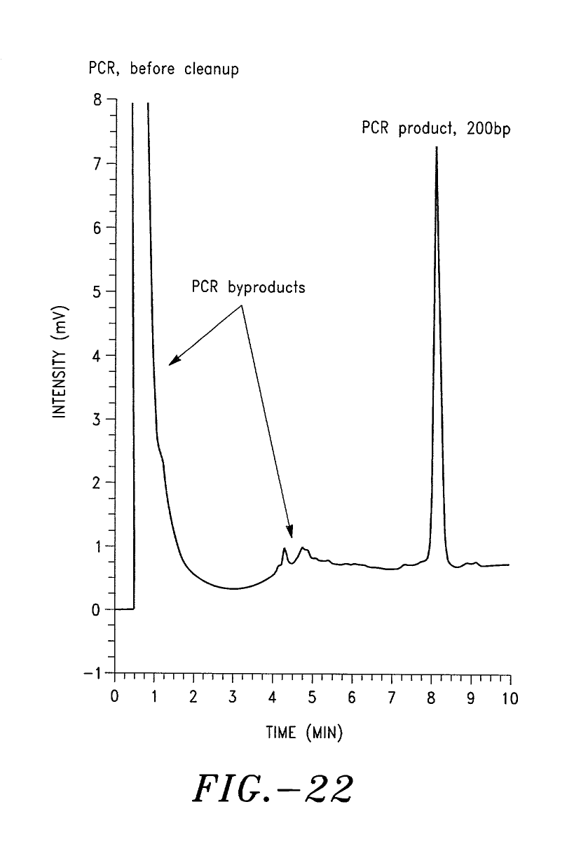

An aliquot of the sample solution was pipetted into separate spin columns and left standing for 5 min. Each vial was centrifuged at 5000 rpm for 5 min. Then 400 μl of freshly prepared aqueous solution containing 38% B (B is an aqueous 25% ACN solution, 0.1 M TEAA) was pipetted into each spin column, each vial was left standing for 5 min, each vial was centrifuged at 5000 rpm for 5 min, and the filtrate was analyzed using the WAVE separation system. The chromatogram obtained for the eluant is shown in FIG. 20 for the pBR322HAE III standard. The treatment procedure was repeated.

The above procedure was repeated, replacing the 38% B solution with 100 μl of a 100% B solution. The chromatogram by analysis with the eluant with the WAVE separation system is shown in FIG. 21 for the pBR322HAE III standard.

This example demonstrates the removal of smaller size fragments from the column while retaining the larger-sized fragments on the column, and subsequent removal of the larger-sized fragments from the column. This is particularly useful for purifying a larger-sized fragment or fragments from smaller size contaminants.

### EXAMPLE 16

### Purification of PCR Product With Spin Column

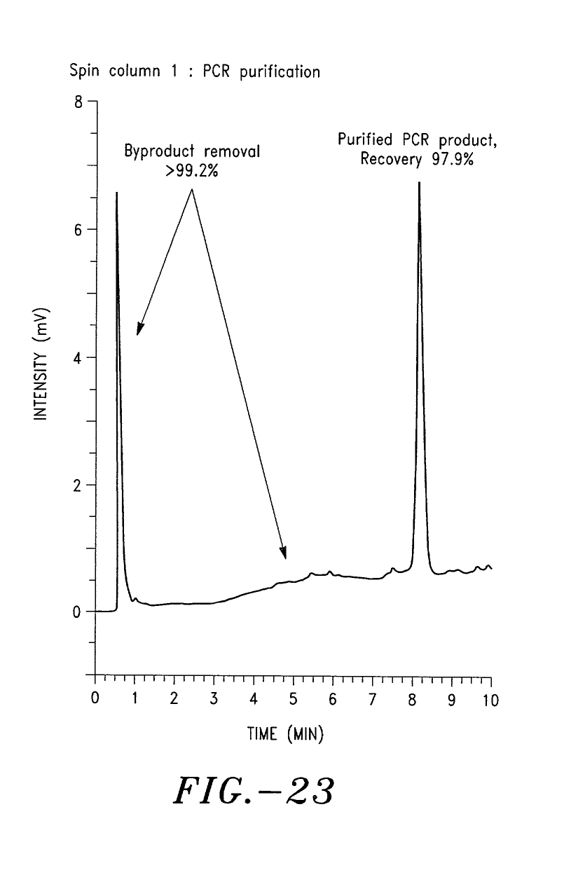

A sample solution was prepared by pipetting 100 μl 0.2 M TEAA onto the spin column and pipetting 100 μl of a 200 bp fragment (p53 exon 6 genomic DNA) which had been amplified by PCR. Separation of the solution was achieved by IP-RP-HPLC using the WAVE nucleic acid analysis system (Transgenomic, Inc., San Jose, Calif.), and shown in FIG. 22.

An aliquot of the sample solution was pipetted into separate spin columns and left standing for 2 min. Each vial was centrifuged at 5000 rpm for 5 min. Then 400 μl of freshly prepared aqueous solution containing 38% B (B is an aqueous 25% ACN solution, 0.1 M TEAA) was pipetted into each spin column, each vial was left standing for 2 min, each vial was centrifuged at 5000 rpm for 5 min, and the filtrate was analyzed using the WAVE separation system. The treatment procedure was repeated.

The above procedure was repeated, replacing the 38% B solution with 100 μl of a 100% B solution. The chromatogram by analyzing the eluant with the WAVE separation system is shown in FIG. 23 for the 200 bp fragment. FIG. 23 shows a purified PCR product recovery of 97.9% and a byproduct removal of greater than 99.2% This example demonstrates the ability of this procedure to elute PCR product with a high recovery and almost complete removal of PCR byproducts.

### EXAMPLE 17

### Purification of Oligonucleotide by Spin Column

An 18-mer oligonucleotide (5′-CGCGCGTTCAGGCTCCGG-3′; SEQ ID NO.: 1) was phosphorylated by reaction with T4 polynucleotide kinase (PNK), using the following standard protocol. 5 μl (1.6 μg) of the single stranded 18-mer oligonucleotide was mixed with 2 μl 10× PNK buffer, 2 μl 10 mM dATP and 10 μl T4 polynucleotide kinase(0.05U/μl) and brought to 20 μl with sterile water. Reactions were incubated for 30 min at 37° C. and were stopped with the addition of 1 μl of 0.5M EDTA. The reaction was purified using a spin column as described above with the following protocol: (1) diluted PNK reaction 1:5 with binding buffer (6% acetonitrile, 0.12M TEAA); (2) added to spin column and centrifuged @ 12000 rpm for 1 minute; (3) discarded flow-through and added 750 μl of binding buffer to the column; (4) centrifuged @ 12000 rpm for 1 minute; (5) discarded flow through vial and placed spin column in a new vial; (6) added 100 μl of Elution buffer (10% acetonitrile, 10 mM Tris-HCl); (7) centrifuged @ 12000 rpm for 1 minute and collected the oligonucleotide-containing eluant.

The eluant and unpurified reaction product were analyzed by IP-RP-HPLC using C-18 alkylated nonporous poly(styrene-divinylbenzene) beads packed in a 50 mm×7.8 mm ID column (DNASEP® cartridge, Transgenomic, Inc., San Jose, Calif.) and using a WAVE® Nucleic Acid Fragment Analysis System (Transgenomic). Buffer A: 0.1 M TEAA, pH 7.0; buffer B: 0.1 TEAA, 25% (v/v) acetonitrile, pH 7.0. The gradient conditions were as follows:

| Time (min) | % B |
|---|---|
| 0.0 | 20 |
| 2.0 | 20 |
| 14 | 32 |
| 15 | 100 |
| 16.5 | 100 |
| 17 | 20 |
| 19 | 20 |

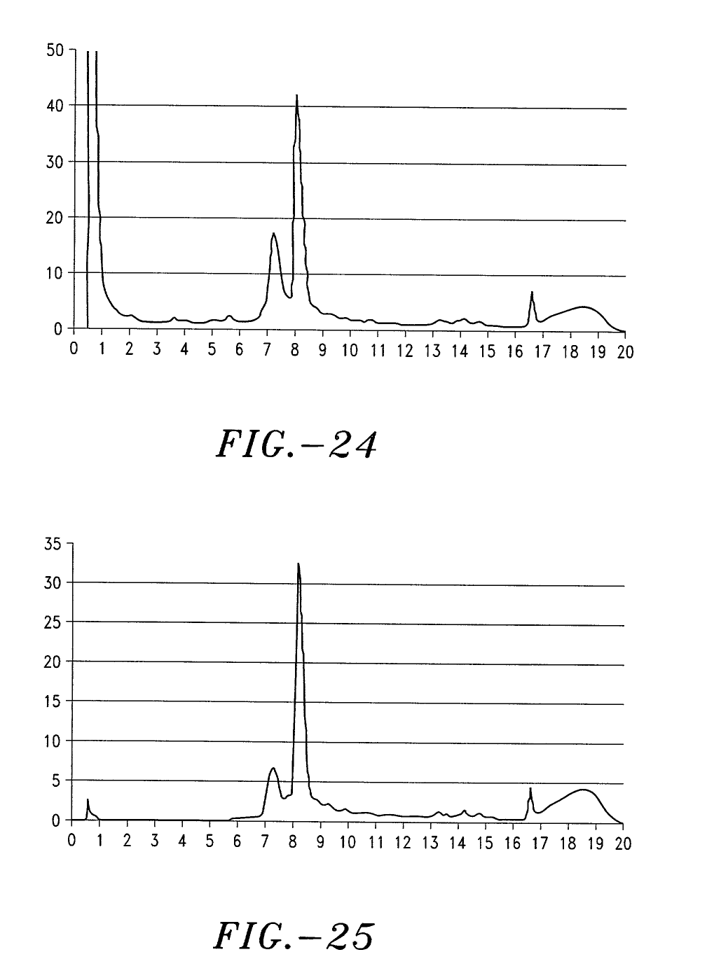

The flow rate was 0.9 mL/min and the column temperature was 50° C. UV detection was performed at 260 nm. The injection volume was 10 μL of the unpurified kinase reaction and 50 μl of the purified kinase reaction. The resulting chromatograms are shown in FIGS. 24 and 25. The two peaks appearing at around 8 minutes are believed to represent the 18-mer oligonucleotide (main peak) and an N-1 mer that occurred during oligonucleotide synthesis and that elutes slightly ahead of the 18-mer. Quantification of the peaks revealed that about 42% of the N-1 mer and 82% of the 19 mer were recovered following spin column purification. It is apparent from comparison of the chromatograms that the spin column removes substantial amounts of the by-products that elute at around 0.5-1 minutes, presumably salts and nucleotides from the PNK reaction. The spin column purification also results in high recovery of the oligonucleotide.

### Example 18

### Purification of a GA Sequencing Ladder and DNA Footprinting Reaction by Spin Column

The oligonucleotides used in this example were synthesized on an Applied Biosystems 394 DNA synthesiser using cyanoethyl phosphoramidite chemistry. Following deprotection, the oligonucleotides were purified using denaturing PAGE, evaporated to dryness and desalted using a Pharmacia NAP 10 column according to the manufacturer's instructions. 5 pmol of labeled synthetic Holliday junction HJ50 was prepared by annealing and purifying the four 50-mer oligonucleotides HJ1, HJ2, HJ3 and

| | |
|---|---|
| HJ4 (HJ1 5′GTCGGATCCTCTAGACAGCTCCATGTTCACTGGCACTGGTAGAATTCGGC | (SEQ ID NO: 2), |
| HJ2 5′-ACGTCATAGACGATTACATTGCTACATGGAGCTGTCTAGAGGATCCGA | (SEQ ID NO: 3); |
| HJ3 5′-(6-FAM)-TGCCGAATTCTACCAGTGCCAGTGCCAGTGATGGACATCTT-TGCCCACGTTGACCC | (SEQ ID NO: 4) and |
| HJ4 5′-(TET)-GGGTCAACGTGGGCAAGATGTCCTAGCAATGTAATCGTCTATGACGTT | (SEQ ID NO: 5)), |

essentially as described in Parsons et al. (1990) J Biol Chem 265:9285-9.

HJ50 was added to a solution of 100 mM Ascorbate (Aldrich), followed by 5 μl of 1.2% H₂O₂ (Aldrich), 10 μl of 20 mM Fe ²⁺/40 mM EDTA (Aldrich) solution was added and rapidly mixed and incubated at room temp for 4 minutes. The reaction was then stopped by the addition of 10 μl of 0.1M thiourea (Sigma) and 0.1M EDTA solutions.

20 μl of this solution was then analyzed using IP-RP-HPLC on a DNASep® column (Transgenomic, Inc.; San Jose, Calif.) under denaturing conditions. Prior to IP-RP-HPLC, the reaction product was purified using a spin-column containing octadecyl modified, nonporous alkylated poly(styrene-divinylbenzene) bead.. The spin columns were first incubated with 500 μl of 0.0025M tBuBr (tetrabutylammonium bromide). A volume of 0.0025M tBuBr equal to the reaction volume was added to the reaction mixtures and then loaded onto the column. The columns were then washed twice with 0.0025M tBuBr containing 2mM EDTA (pH 8.0). The DNA fragments were then eluted using 70% acetonitrile and load onto the DNAsep® column.

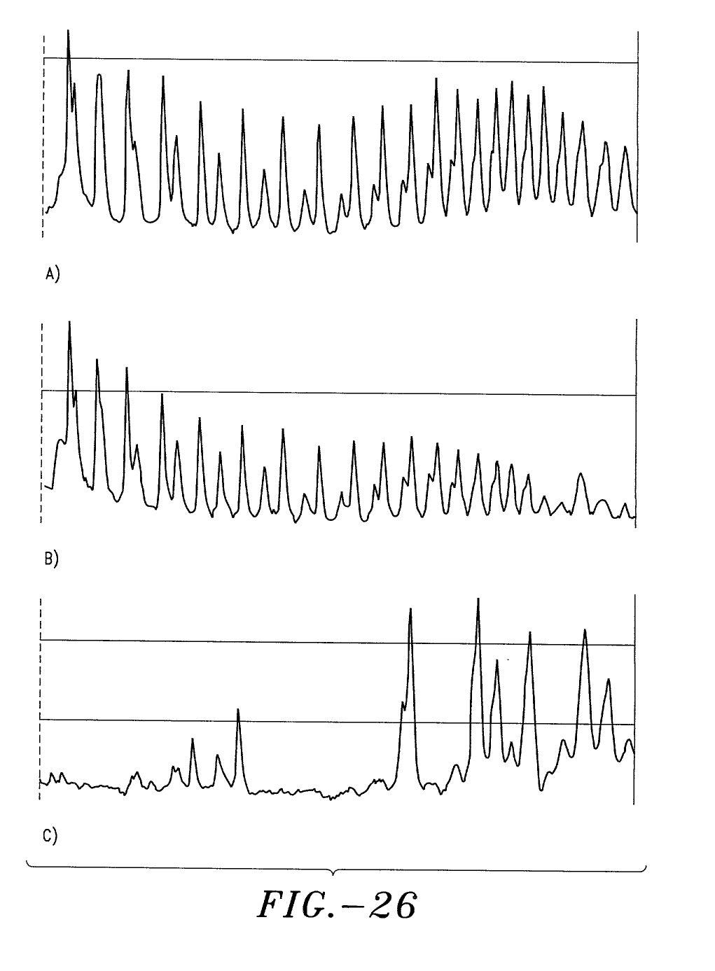

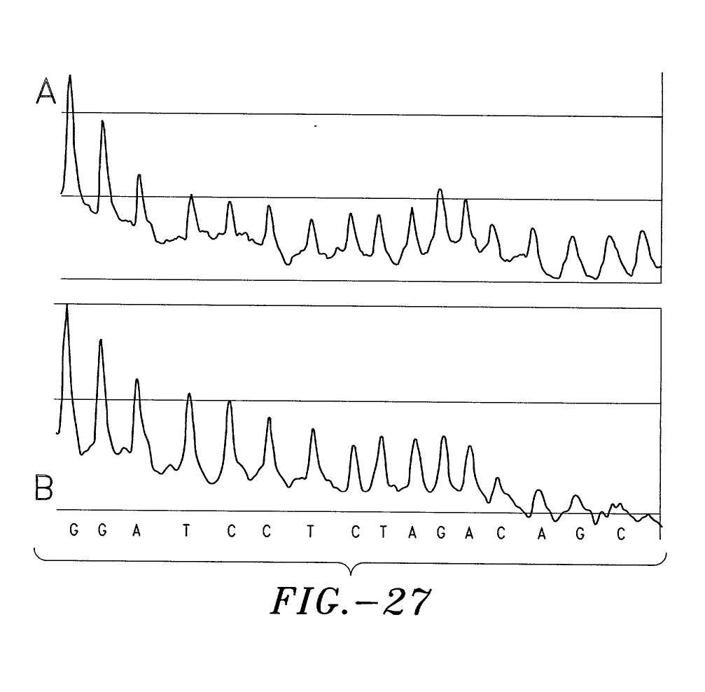

The chromatographic separation was controlled by a WAVE® fragment analysis system (Transgenomic, Inc.; San Jose, Calif.) at 70° C. using fluorescence detection at the appropriate excitation and emission wavelengths (FAM: Ex 494, Em 525; TET: Ex 521, Em 536). The following elution gradient was employed: Buffer A 0.0025 M Tetrabutylammonium bromide (Fisher HPLC), 0.1% acetonitrile, Buffer B 0.0025M, Tetrabutylammonium bromide, 70% acetonitrile. The run was initiated at 30% buffer B, the gradient was extended to 50% buffer B over 12 minutes at a flow rate of 0.9 ml/min, followed by an extension to 60% buffer B over 18 minutes at a flow rate of 0.9 ml/min. The chromatogram (FIG. 26a) shows the effect of hydroxyl radical cleavage of FAM-labeled strand HJ3 in the absence of protein.

The experiment was repeated as above, this time with the inclusion 1 μM E. coli RuvA, a Holliday junction-binding protein. RuvA was purified as described in Sedelnikova et al. (1997) Acta. Cryst. D53:122-24. FIG. 26b shows that the protein protected strand HJ3 from cleavage in the right portion of the chromatogram.

In order to phase the chromatogram, the labeled DNA was used to generate a G+A sequencing ladder by the method of Belikokv and Wieslander (supra). 10 μl of 3% diphenylamine (Aldrich) in formic acid (Aldrich) was added to 75 pmol of the labeled DNA. The reaction volume was then made up to 20 μl with MilliQ water and incubated at room temp for 10 minutes. The reaction was stopped by the addition of 100 μl 0.3M sodium acetate (pH 5.5) and the mixture was extracted three times with water saturated ether. The sample was then placed in a vacuum dryer to remove traces of ether and precipitated by the addition of 3 volumes of ethanol and placed at −20° C. for 30 minutes. The DNA was then precipitated for 15 mins at 15, 000 g, re-suspended in Milli Q water (20 μl) and purified by spin-column as described above. 5 μl was then analyzed by IP-RP-HPLC using the conditions described above (FIG. 26c).

The above procedure was repeated, the only difference being that the TET-labeled HJ4 strand was detected. The resulting chromatograms for the control reaction and the RuvA-including reaction are presented in FIGS. 27a and 27b, respectively.

### Example 19

### Use of a Spin Column to Separate Genomic DNA From RNA

The following example describes the isolation and collection of both RNA and genomic DNA (gDNA) from a single sample. The procedure involves binding a sample of RNA and gDNA to a column, a wash step to remove impurities, elution of RNA in an RNA isolation butter, and finally elution of gDNA with a gDNA isolation buffer.

Prior to application of the sample the spin column is washed of multivalent cations and RNAse activity as described in Example 13. A sample containing RNA and gDNA is diluted 1:5 in binding buffer (6.0% ACN/0.12 M TEAA), loaded on the spin column, and centrifuged at 7000 rpm for 1 min. 600 μL of Wash buffer (10.0% ACN/0.12M TEAA) is added to the spin column and the column is spun at 7000 rpm for 1 min. Next, 100 μL of RNA Isolation buffer (18.0% ACN/0.1 M TEAA) is added to the spin column and the column is spun at 7000 rpm for 1 min, where the RNA comes out in the eluant. Finally, 100 μL of gDNA Isolation buffer (25.0% ACN) is added to the spin column and the column is spun at 7000 rpm for 1 min, where the gDNA comes out in the eluant.

Obviously, numerous modifications and variations of the present invention are possible in light of the above teachings. It is therefore to be understood that within the scope of the appended claims, the invention may be practiced otherwise than as specifically described herein.

The invention claimed is:

1. A non-HPLC chromatographic method for purifying a target polynucleotide comprising the steps of:
a) applying the target polynucleotide to a separation medium having a non-polar separation surface in the presence of a counterion agent, whereby the polynucleotide is bound to the separation medium;
b) eluting the target polynucleotide from the separation medium by passing through the separation medium an elution solution containing a concentration of organic solvent sufficient to elute the target polynucleotide from the separation medium; and
c) collecting the eluted target polynucleotide.

2. The method of claim 1, wherein the target polynucleotide is applied to the separation medium as a component of a loading solution containing a non-target molecule.

3. The method of claim 2, wherein the non-target molecule is not bound to the separation medium in the presence of the loading solution, and is thereby eluted from the separation medium and separated from the target polynucleotide by passing the loading solution through the separation medium.

4. The method of claim 2 wherein the non-target molecule is bound to the separation medium in the presence of the loading solution, and including an additional step between steps (a) and (b) of eluting the non-target molecule from the separation medium by passing through the separation medium a wash solution containing a counterion agent and a concentration of organic solvent sufficient to elute the non-target molecule, but insufficient to elute the target polynucleotide from the separation medium, whereby the non-target molecule is separated from the target polynucleotide.

5. The method of claim 2 wherein the non-target molecule remains bound to the separation medium in the presence of the elution solution, and is thereby separated from the target polynucleotide during the elution step.

6. The method of any of claims 1-5, wherein the separation medium has a nonpolar separation surface that is substantially free of multivalent cations that are capable of interfering with polynucleotide separations.

7. The method of claim 6, wherein the solutions used are substantially free of multivalent cations capable of interfering with polynucleotide separations.

8. The method of claim 1, wherein the non-target molecule is a polynucleotide.

9. The method of claim 8, wherein the polynucleotide is double-stranded DNA.

10. The method of claim 8, wherein the polynucleotide is RNA.

11. The method of claim 8, wherein the polynucleotide is single-stranded DNA.

12. The method of claim 11, wherein the DNA is an oligonucleotide.

13. The method of claim 1, wherein a mixture of polynucleotide fragments of varying nucleotide length is applied to the separation medium, and wherein the elution solution contains a concentration of organic solvent that has been predetermined to elute polynucleotide fragments falling within a defined range of nucleotide lengths, whereby polynucleotide fragments falling within the defined range of nucleotide lengths are eluted from the separation medium and thereby separated from other polynucleotides of the mixture.

14. The method of claim 13, wherein the polynucleotide fragments are double-stranded DNA fragments.

15. The method of claim 13, wherein the polynucleotide fragments are single-stranded DNA fragments.

16. The method of claim 13, wherein the polynucleotide fragments are RNA fragments.

17. The method of claim 1, wherein the polynucleotide is eluted from separation medium that is supported in a spin column.

18. The method of claim 17, wherein the separation medium is in communication with an upper solution input chamber and a lower eluant receiving chamber, wherein the loading solution containing the polynucleotide and a counterion agent is applied to the separation medium by introducing the solution into the upper solution input chamber and centrifuging the spin column under conditions where the polynucleotide substantially binds to the separation medium, wherein the elution solution is passed through the separation medium by centrifugation of the spin column, and wherein the eluted polynucleotide is collected in the lower eluant receiving chamber.

19. The method of claim 1, wherein the polynucleotide is eluted from separation medium that is supported in a vacuum tray separation device.

20. The method of claim 1, wherein the separation medium comprises particles selected from the group consisting of silica, silica carbide, silica nitrite, titanium oxide, aluminum oxide, zirconium oxide, carbon, insoluble polysaccharide, and diatomaceous earth, the particles having separation surfaces which are coated with a hydrocarbon or non-polar hydrocarbon substituted polymer, or have substantially all polar groups reacted with a non-polar hydrocarbon or substituted hydrocarbon group, wherein the surfaces are non-polar.

21. The method of claim 6, wherein the separation medium comprises polymer beads having an average diameter of 0.5 to 100 microns, the beads being unsubstituted polymer beads or polymer beads substituted with a moiety selected from the group consisting of hydrocarbon having from one to 1,000,000 carbons.

22. The method of claim 1, wherein the separation medium comprises a monolith.

23. The method of claim 1, wherein the separation medium comprises capillary channels.

24. The method of claim 1, wherein the separation medium has been subjected to acid wash treatment to remove any residual surface metal contaminants.

25. The method of claim 1, wherein the separation medium has been subjected to treatment with a multivalent cation binding agent.

26. The method of claim 1, wherein the organic solvent is selected from the group consisting of alcohol, nitrile, dimethylformamide, tetrahydrofuran, ester, ether, and mixtures of one or more thereof.

27. The method of claim 26, wherein the organic solvent comprises acetonitrile.

28. The method of claim 1, wherein the counterion agent is selected from the group consisting of lower alkyl primary amine, lower alkyl secondary amine, lower alkyl tertiary amine, lower trialkylammonium salt, quaternary ammonium salt, and mixtures of one or more thereof.

29. The method of claim 28, wherein the counterion agent is selected from the group consisting of octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyidiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, tetrabutylammonium acetate, triethylammonium hexafluoroisopropyl alcohol, and mixtures of one or more thereof.

30. The method of claim 29, wherein the counterion agent is tetrabutylammonium acetate.

31. The method of claim 29, wherein the counterion agent is triethylammonium acetate.

32. The method of claim 1, wherein the target polynucleotide is applied to the separation medium under denaturing conditions.

33. The method of claim 1, wherein a sample containing RNA and genomic DNA is separated into a RNA-containing fraction and a genomic DNA-containing fraction.

34. A device for purifying a target polynucleotide comprising a tube having:
a) an upper solution input chamber;
b) a lower eluant receiving chamber; and
c) a fixed unit of separation medium supported therebetween, wherein the separation medium has a nonpolar separation surface that is substantially free of multivalent cations that are capable of interfering with polynucleotide separations.

35. The device of claim 32, wherein the separation medium is selected from the group consisting of beads, capillary channels and monolith structure.

36. The device of claim 33, wherein the fixed unit of separation medium comprise a fixed bed of separation medium particles.

37. The device of claim 34, wherein the separation medium particles are selected from the group consisting of organic polymer and inorganic particles having a nonpolar surface.

38. The device of claim 32, wherein the lower chamber is closed.

39. The device of claim 32, wherein the lower chamber has an open bottom portion.

40. The device of claim 37 in combination with an eluant container shaped to receive said lower chamber.

41. The device of claim 38 wherein the eluant chamber is a centrifuge vial.

42. The device of claim 38 wherein the cylinder is a member of an array of cylinders and the eluant container is a member of an array of eluant containers, and the array of cylinders and array of containers have matching configurations.

43. A separation system comprising a multicavity separation plate having outer sealing edges, a multiwell collection plate and a vacuum system having a separation plate sealing means forming a sealed engagement with the outer sealing edges of the multicavity separation plate and a vacuum cavity receiving the multiwell collection plate; the multicavity separation plate including an array of tubes, each tube having an upper solution input chamber, a lower eluant receiving chamber with an bottom opening therein, and a fixed unit of separation medium supported therein, the separation medium having nonpolar separation surfaces that are free from multivalent cations that are capable of interfering with polynucleotide separations; the multiwell collection plate having collection wells which are positioned to receive liquid from the bottom opening of the lower eluant receiving chamber.

44. The separation system of claim 43, wherein the separation medium is selected from the group consisting of beads, capillary channels and monolith structures.

45. The separation system of claim 44, wherein the fixed unit of separation medium comprise a fixed bed of separation medium particles.

46. The separation system of claim 45, wherein the separation medium particles are selected from the group consisting of organic polymer and inorganic particles having a nonpolar surface.
